SEQUENCE LISTING (47 sequences)

SEQ ID NO: 1 - DNA, 43 bp, Artificial Sequence
  Enolase 1 (ENO1) forward primer
tatatgggcccactagtcttctaggcgggttatctactgatcc

SEQ ID NO: 2 - DNA, 72 bp, Artificial Sequence
  Enolase 1 (ENO1) overlap primer
ggactagaaggcttaatcaaaagcggcgcgccggatccttaattaagtgtgttgataagcagttgcttggtt

SEQ ID NO: 3 - DNA, 42 bp, Artificial Sequence
  Enolase 1 (ENO1) reverse primer
gctacgaattcgcggccgccgtcgaacaacgttctattagga

SEQ ID NO: 4 - DNA, 33 bp, Artificial Sequence
  Phosphoglycerate kinase (PGK) forward primer
tatatgggccctccctccttcttgaattgatgt

SEQ ID NO: 5 - DNA, 79 bp, Artificial Sequence
  Phosphoglycerate kinase (PGK) overlap primer
gatctatcgatttcaattcaattcaatggcgcgccggatccttaattaatgtaaaaagtagataattacttccttgatg

SEQ ID NO: 6 - DNA, 31 bp, Artificial Sequence
  Phosphoglycerate kinase (PGK) reverse primer
cttaggaattctttcgaaacgcagaattttc

SEQ ID NO: 7 - DNA, 30 bp, Artificial Sequence
  Kanamycin (kanMX) forward primer
gatccgaattcgtttagcttgcctcgtccc

SEQ ID NO: 8 - DNA, 30 bp, Artificial Sequence
  Kanamycin (kanMX) reverse primer
cagtcgactagttttcgacactggatggcg

SEQ ID NO: 9 - DNA, 33 bp, Artificial Sequence
  Zeocin (Zeo) forward primer
gcgctagaattccccacacaccatagcttcaaa

SEQ ID NO: 10 - DNA, 41 bp, Artificial Sequence
  Zeocin (Zeo) reverse primer
ccgcatactagtaattcagcttgcaaattaaagccttcgag

SEQ ID NO: 11 - DNA, 41 bp, Artificial Sequence
  Beta-glucosidase I (BGLI) forward primer
gccgccttaattaaaaacaaaatggtctccttcacctccct

SEQ ID NO: 12 - DNA, 38 bp, Artificial Sequence
  Beta-glucosidase I (BGLI) reverse primer
cggttggatccaatagtaaacaggacagatgtcttgat

SEQ ID NO: 13 - DNA, 37 bp, Artificial Sequence
  Delta2 forward primer
agtcgcggccgctgttggaataaaaatccactatcgt

SEQ ID NO: 14 - DNA, 40 bp, Artificial Sequence
  Delta2 reverse primer
gcgcccgcggtgagatatatgtgggtaattagataattgt

SEQ ID NO: 15 - DNA, 18 bp, Artificial Sequence
  M13 forward primer
tcccagtcacgacgtcgt

SEQ ID NO: 16 - DNA, 19 bp, Artificial Sequence
  M13 reverse primer
ggaaacagctatgaccatg

SEQ ID NO: 17 - DNA, 27 bp, Artificial Sequence
  Phosphoglycerate kinase (PGK) seq forward primer
tctttttctcttttttacagatcatca

SEQ ID NO: 18 - DNA, 27 bp, Artificial Sequence
  Enolase 1 (ENO1) seq forward primer
tccttctagctatttttcataaaaaac

SEQ ID NO: 19 - DNA, 20 bp, Artificial Sequence
  Endoglucase I (EGI) detection forward primer
cgctagtggtgttacgacga

SEQ ID NO: 20 - DNA, 20 bp, Artificial Sequence
  Endoglucanase I (EGI) detection reverse primer
ctccaagtctgcactggaca

SEQ ID NO: 21 - DNA, 20 bp, Artificial Sequence
  Beta-glucosidase I (BGLI) detection forward primer
gagcccgcattattatccaa

SEQ ID NO: 22 - DNA, 20 bp, Artificial Sequence
  Beta-glucosidase I (BGLI) detection reverse primer
caaagtcagcgaatcgaaca

SEQ ID NO: 23 - DNA, 20 bp, Artificial Sequence
  Cellobiohydrolase I (CBHI) detection forward primer
agacggttgtgactggaacc

SEQ ID NO: 24 - DNA, 20 bp, Artificial Sequence
  Cellobiohydrolase I (CBHI) detection reverse primer
caacttgagctggaacacca

SEQ ID NO: 25 - DNA, 20 bp, Artificial Sequence
  Cellobiohydrolase II (CBHII) detection forward primer
cagagactgtgctgctttgg

SEQ ID NO: 26 - DNA, 20 bp, Artificial Sequence
  Cellobiohydrolase II (CBHII) detection reverse primer
ggatctaccttggtcggtga

SEQ ID NO: 27 - DNA, 358 bp, Saccharomyces cerevisiae
  exon (1)..(358)
agtcggtacctgttggaataaaaatccactatcgtctatcaactaatagttatattatcaatatattatcatatacggtgttaagatgatgacataagttatgagaagctgtcatcgatgttagaggaagctgaaacgcaaggattgataatgtaataggatcaatgaatataaacatataaaacggaatgaggaataatcgtaatattagtatgtagaaatatagattccattttgaggattcctatatcctcgaggagaacttctagtatattctgtatacctaatattatagcctttatcaacaatggaatcccaacaattatctaattacccacatatatctcagggcccgcgc

SEQ ID NO: 28 - DNA, 1354 bp, Trichoderma reesei
  exon (1)..(1354)
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgaccgcatgtgattctgccggttgtggtttcaacccttatggttctggttataagagttactacggtccgggagacaccgtggatacgtcaaagaccttcactataatcactcagtttaacacagataacggatctccgagtggtaatttggtgagtattactaggaaatatcagcagaacggtgttgatattccgtccgcgcagccaggcggtgacactatatctagctgtccttccgccagtgcctatggcggacttgctacaatgggtaaggcattgtcctcaggtatggtcctagtattttctatttggaatgataattcacaatacatgaattggctggattctggtaatgcaggcccttgctcctctacagaaggtaacccaagcaatatactagctaataacccaaatactcatgttgtctttagtaatattagatggggcgatataggtagcactacgaacagtaccgcacctcctcctccacctgctagctccacgacattttccactactagaaggtccagcactaccagctcatcaccatcttgtactcaaacccattggggacagtgtggtggtataggttacagcggttgcaaaacttgcacatctggtactacatgccaatacagtaatgactattactcacaatgtttaccaggtgctgcgtcaagttcaagtagtggatcc

SEQ ID NO: 29 - DNA, 2520 bp, Trichoderma reesei
  exon (1)..(2520)
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaacctcctctactccaggtgctgtcagaggttcctgttctacctcttctggtgttccagctcaagttgaatctcaatctccaaacgctaaggtcactttctccaacatcaagttcggtccaatcggttccactggtaatccatctggtggaaaccctccaggtggtaacagaggtactaccactactcgtaggccagctactacaactggttcttccccaggcccaacccaatcccactacggtcaatgtggtggtatcggttactctggtccaaccgtctgtgcttctggtactacctgtcaagttttaaacccatactactctcaatgtttgcctggtgctgcttccagttcatctagtggatccggtggcggtggatctggaggaggcggttcttggtctcacccacaatttgaaaagggtggagaaaacttgtactttcaaggcggtggtggaggttctggcggaggtggctccggctcagctatctctcaaatcaccgacggtcaaatccaagccactaccacagctaccactgaagctacaactaccgctgctccttcatctactgttgaaactgtttctccatcttccaccgaaaccatctctcaacaaaccgaaaacggtgctgctaaggctgctgttggtatgggtgctggtgctttggctgctgctgctatgttgttgtagggcgcgcc

SEQ ID NO: 30 - DNA, 3314 bp, Trichoderma reesei
  exon (1)..(3314)
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaaccgagtcccggggtcccattagaagaaagacaagcctgctcctctgtttggggtcaatgtggtggtcaaaactggtctggtccaacttgttgtgcttccggttctacctgtgtttactccaacgactactattcccaatgtttgccaggtgctgcttcctcttcctcttcaactagagctgcttctacaacttctagggtctccccaaccacttccagatcctcttctgctactccaccaccaggttctactaccactagagttccaccagtcggttccggtactgctacttactctggtaaccctttcgtcggtgttactccatgggctaacgcttactacgcttctgaagtttcttctttggctatcccatctttgactggtgctatggctaccgctgctgctgctgtcgccaaagttccatccttcatgtggttggacaccttggacaaaactccattaatggaacaaaccttggcagacataaggactgctaacaagaacggcggtaactacgctggtcaatttgttgtgtacgacttgccagacagagactgtgctgctttggcttccaacggtgaatactccatcgctgacggtggtgtcgccaagtacaagaactacattgataccattagacaaatcgttgtcgaatactctgacatcagaaccttgttagtcatcgaaccagattctttagccaatttagtcaccaacttgggtactccaaagtgtgctaacgctcaatctgcctacttagaatgtatcaattatgcagttacccaattgaacttgccaaacgttgctatgtacttggacgctggtcacgccggttggttgggttggccagctaaccaagacccagccgctcaattattcgccaacgtttacaagaatgcctcttctcctagagccttgcgtggtttggctactaacgtcgctaactacaacggttggaacatcacttctccaccatcttacacccaaggtaacgctgtttacaacgaaaagttgtacattcacgctatcggtccattattggctaaccatggttggtctaacgccttcttcatcaccgaccaaggtagatccggtaaacaaccaactggtcaacaacaatggggtgattggtgtaacgtcatcggtactggtttcggtatcagaccatccgctaacactggtgattccttgttggattccttcgtctgggttaagccaggtggtgaatgtgatggcacctctgattcctctgctccaagattcgattcccactgcgccttgccagacgctttgcaaccagccccacaagctggtgcatggttccaagcttactttgtccaattgttgaccaacgctaacccatctttcttgggatccggtggcggtggatctggtggaggcggttctcatcaccaccatcatcacggtggcgaaaacttgtactttcaaggcggcggtggaggtagtggaggaggtggctccggctcagct

SEQ ID NO: 31 - DNA, 2996 bp, Artificial Sequence
  68 C-terminal AA from CWP2p
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaaccgagtcccggggtcccattagaagaaagacaagcctgctcctctgtttggggtcaatgtggtggtcaaaactggtctggtccaacttgttgtgcttccggttctacctgtgtttactccaacgactactattcccaatgtttgccaggtgctgcttcctcttcctcttcaactagagctgcttctacaacttctagggtctccccaaccacttccagatcctcttctgctactccaccaccaggttctactaccactagagttccaccagtcggttccggtactgctacttactctggtaaccctttcgtcggtgttactccatgggctaacgcttactacgcttctgaagtttcttctttggctatcccatctttgactggtgctatggctaccgctgctgctgctgtcgccaaagttccatccttcatgtggttggacaccttggacaaaactccattaatggaacaaaccttggcagacataaggactgctaacaagaacggcggtaactacgctggtcaatttgttgtgtacgacttgccagacagagactgtgctgctttggcttccaacggtgaatactccatcgctgacggtggtgtcgccaagtacaagaactacattgataccattagacaaatcgttgtcgaatactctgacatcagaaccttgttagtcatcgaaccagattctttagccaatttagtcaccaacttgggtactccaaagtgtgctaacgctcaatctgcctacttagaatgtatcaattatgcagttacccaattgaacttgccaaacgttgggatccggaggtggttcaggaggtggtgggtctgcttggcatccacaatttggaggaggcggtggtgaaaatctgtatttccagggaggcggaggtgattacaaggatgacgacaaaggaggtggtggatcaggaggtggtggctccggctcagctattagccaaataactgatggtcaaatacaagcaactacaacagcaacaaccgaagctactaccacagccgcgccttcttcaactgttgagactgttagtccttcctccacggaaacgatttctcaacagactgaaaacggtgcagccaaagcagcagtcggcatgggtgccggagccctagcagctgcagcaatgcttttgtaaggcgcgcc

SEQ ID NO: 32 - DNA, 2776 bp, Trichoderma reesei
  exon (1)..(2776)
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaaccgagtcccggggtcccattagaagaaagacaagcctgctcctctgtttggggtcaatgtggtggtcaaaactggtctggtccaacttgttgtgcttccggttctacctgtgtttactccaacgactactattcccaatgtttgccaggtgctgcttcctcttcctcttcaactagagctgcttctacaacttctagggtctccccaaccacttccagatcctcttctgctactccaccaccaggttctactaccactagagttccaccagtcggttccggtactgctacttactctggtaaccctttcgtcggtgttactccatgggctaacgcttactacgcttctgaagtttcttctttggctatcccatctttgactggtgctatggctaccgctgctgctgctgtcgccaaagttccatccttcatgtggttggacaccttggacaaaactccattaatggaacaaaccttggcagacataaggactgctaacaagaacggcggtaactacgctggtcaatttgttgtgtacgacttgccagacagagactgtgctgctttggcttccaacggtgaatactccatcgctgacggtggtgtcgccaagtacaagaactacattgataccattagacaaatcgttgtcgaatactctgacatcagaaccttgttagtcatcgaaccagattctttagccaatttagtcaccaacttgggtactccaaagtgtgctaacgctcaatctgcctacttagaatgtatcaattatgcagttacccaattgaacttgccaaacgttggaattcttaattaaaaacaaaatggtctccttcacctccctgctggccggcgttgccgctatctctggtgtcctagcagcccctgccgcagaagttgaacctgtcgcagttgagaaacgtgaggccgaagcagaagctcccgggactc

SEQ ID NO: 33 - DNA, 2932 bp, Artificial Sequence
  Mfalpha pre-pro secretion signal and spacer
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaaccgagtcccggggtcccattagaagaaagacaagcctgctcctctgtttggggtcaatgtggtggtcaaaactggtctggtccaacttgttgtgcttccggttctacctgtgtttactccaacgactactattcccaatgtttgccaggtgctgcttcctcttcctcttcaactagagctgcttctacaacttctagggtctccccaaccacttccagatcctcttctgctactccaccaccaggttctactaccactagagttccaccagtcggttccggtactgctacttactctggtaaccctttcgtcggtgttactccatgggctaacgcttactacgcttctgaagtttcttctttggctatcccatctttgactggtgctatggctaccgctgctgctgctgtcgccaaagttccatccttcatgtggttggacaccttggacaaaactccattaatggaacaaaccttggcagacataaggactgctaacaagaacggcggtaactacgctggtcaatttgttgtgtacgacttgccagacagagactgtgctgctttggcttccaacggtgaatactccatcgctgacggtggtgtcgccaagtacaagaactacattgataccattagacaaatcgttgtcgaatactctgacatcagaaccttgttagtcatcgaaccagattctttagccaatttagtcaccaacttgggtactccaaagtgtgctaacgctcaatctgcctacttagaatgtatcaattatgcagttacccaattgaacttgccaaacgttggaattcttaattaaaaacaaaatgagatttccatcaatatttacagcagttttgtttgcggcgagttcagcccttgcagcacccgtcaataccacgacggaggatgagacagcccagatcccagcagaggctgtgataggatatttagacctggaaggcgattttgatgtggccgtattaccgttttctaactctacgaataatggattgttatttattaatactacaattgcctctatagccgcaaaggaagaaggggtgtctttagataagagagaagctgaggctgaagcccccgggactc

SEQ ID NO: 34 - DNA, 2812 bp, Artificial Sequence
  Hybrid killer toxin
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaaccgagtcccggggtcccattagaagaaagacaagcctgctcctctgtttggggtcaatgtggtggtcaaaactggtctggtccaacttgttgtgcttccggttctacctgtgtttactccaacgactactattcccaatgtttgccaggtgctgcttcctcttcctcttcaactagagctgcttctacaacttctagggtctccccaaccacttccagatcctcttctgctactccaccaccaggttctactaccactagagttccaccagtcggttccggtactgctacttactctggtaaccctttcgtcggtgttactccatgggctaacgcttactacgcttctgaagtttcttctttggctatcccatctttgactggtgctatggctaccgctgctgctgctgtcgccaaagttccatccttcatgtggttggacaccttggacaaaactccattaatggaacaaaccttggcagacataaggactgctaacaagaacggcggtaactacgctggtcaatttgttgtgtacgacttgccagacagagactgtgctgctttggcttccaacggtgaatactccatcgctgacggtggtgtcgccaagtacaagaactacattgataccattagacaaatcgttgtcgaatactctgacatcagaaccttgttagtcatcgaaccagattctttagccaatttagtcaccaacttgggtactccaaagtgtgctaacgctcaatctgcctacttagaatgtatcaattatgcagttacccaattgaacttgccaaacgttggaattcttaattaaaaacaaaatgaatatattttatattttcctatttcttttatcatttgtgcagggatcattaaattgtacattaagagattcacaacaaaagtctttagtaatgtcaggtccatatgaattaaaagcatcccttgataaaagggaagccgaagccgaagctcccgggactc

SEQ ID NO: 35 - DNA, 2881 bp, Artificial Sequence
  Kjeldsen synthetic and spacer
gagtcccgggcaacaaccaggaacatcaacaccagaagtccatccaaagttaacaacctataaatgtactaagagtggagggtgtgtagcgcaggacacaagtgtggtcttagactggaattatcgttggatgcatgatgccaattataattcctgtactgttaacggcggtgttaacactacgttatgccccgatgaagcgacttgtggtaagaattgttttattgaaggggttgactacgccgctagtggtgttacgacgagtgggtcatccttgacgatgaatcaatacatgccttcttctagtggtgggtattcctctgtgtctccaaggctgtatttattggattccgatggggaatatgttatgttaaaattaaatgggcaagaactgagttttgatgtggatctatctgcattaccttgtggagaaaatggtagtctttatttatcacaaatggacgaaaacggcggagccaatcagtacaatacagctggtgctaattatggttcaggctattgtgatgctcaatgtccagtgcagacttggaggaatggcaccttaaacacatcacatcaaggattttgctgtaacgaaatggacatattagaaggtaattcaagagctaatgcactaactccgcactcttgtactgcgagtcccgggcaatccgcttgtaccctacaatccgaaactcacccaccattgacctggcaaaagtgttctagcggtggaacttgtactcaacaaactggttctgttgttatcgacgctaactggagatggacacacgccactaactcttctaccaactgttacgacggtaacacttggtcttccactttatgtccagataacgaaacttgtgctaagaattgctgtttggacggtgccgcctacgcttctacctacggtgttaccacctccggtaactccttgtctattggtttcgtcactcaatccgctcaaaagaacgttggtgctagattgtacttgatggcttctgacactacttatcaagaatttactttgttgggtaacgaattttctttcgatgttgacgtttcccaattgccatgtggcttgaacggtgctttgtactttgtctctatggatgctgacggtggtgtttctaagtacccaactaacactgccggtgctaagtacggtactggttactgtgattctcaatgtccacgtgacttgaagttcattaacggtcaagccaacgtcgaaggttgggaaccatcctccaacaacgctaacaccggtatcggtggtcacggttcctgttgttccgaaatggacatctgggaagctaacagtatttctgaagctttgacaccacacccatgcaccactgtcggtcaagaaatttgtgaaggtgatggatgtggtggaacctactctgataacagatacggtggtacttgtgacccagacggttgtgactggaacccatacagattgggtaacacttctttctatggtccaggttcttctttcaccttggataccaccaagaagttgactgttgttacccaattcgaaacttctggtgctatcaacagatactacgttcaaaacggtgtcaccttccaacaaccaaacgctgaattgggttcttactctggtaatgaattgaacgacgactactgtaccgctgaagaagctgaatttggtggttcctctttctccgacaagggtggtttgacccaattcaagaaggctacctccggtggtatggttttggttatgtccttgtgggatgattactacgcaaacatgttatggttagacagtacttacccaactaacgaaaccgagtcccggggtcccattagaagaaagacaagcctgctcctctgtttggggtcaatgtggtggtcaaaactggtctggtccaacttgttgtgcttccggttctacctgtgtttactccaacgactactattcccaatgtttgccaggtgctgcttcctcttcctcttcaactagagctgcttctacaacttctagggtctccccaaccacttccagatcctcttctgctactccaccaccaggttctactaccactagagttccaccagtcggttccggtactgctacttactctggtaaccctttcgtcggtgttactccatgggctaacgcttactacgcttctgaagtttcttctttggctatcccatctttgactggtgctatggctaccgctgctgctgctgtcgccaaagttccatccttcatgtggttggacaccttggacaaaactccattaatggaacaaaccttggcagacataaggactgctaacaagaacggcggtaactacgctggtcaatttgttgtgtacgacttgccagacagagactgtgctgctttggcttccaacggtgaatactccatcgctgacggtggtgtcgccaagtacaagaactacattgataccattagacaaatcgttgtcgaatactctgacatcagaaccttgttagtcatcgaaccagattctttagccaatttagtcaccaacttgggtactccaaagtgtgctaacgctcaatctgcctacttagaatgtatcaattatgcagttacccaattgaacttgccaaacgttggaattcttaattaaaaacaaaatgaagttgaagactgttaggtcagccgttttgagtagtttatttgcctctcaagtcttgggtcaaccaattgatgatacggaaagtaataccacttcagttaatttgatggctgacgatacggaatctaggtttgcaacgaacacgaccttagctctagatgttgtgaatttaatttcaatggctaaaagagaagaggctgaagctgaggcggagcccaagcccgggactc

SEQ ID NO: 36 - protein, 915 aa, Saccharomycopsis fibuliga
MVSFTSLLAGVAAISGVLAAPAAEVEPVAVEKREAEAEAMLMIVQLLVFALGLAVAVPIQNYTQSPSQRDESSQWVSPHYYPTPQGGRLQDVWQEAYARAKAIVGQMTIVEKVNLTTGTGWQLDPCVGNTGSVPRFGIPNLCLQDGPLGVRFADFVTGYPSGLATGATFNKDLFLQRGQALGHEFNSKGVHIALGPAVGPLGVKARGGRNFEAFGSDPYLQGTAAAATIKGLQENNVMACVKHFIGNEQEKYRQPDDINPATNQTTKEAISANIPDRAMHALYLWPFADSVRAGVGSVMCSYNRVNNTYACENSYMMNHLLKEELGFQGFVVSDWGAQLSGVYSAISGLDMSMPGEVYGGWNTGTSFWGQNLTKAIYNETVPIERLDDMATRILAALYATNSFPTEDHLPNFSSWTTKEYGNKYYADNTTEIVKVNYNVDPSNDFTEDTALKVAEESIVLLKNENNTLPISPEKAKRLLLSGIAAGPDPIGYQCEDQSCTNGALFQGWGSGSVGSPKYQVTPFEEISYLARKNKMQFDYIRESYDLAQVTKVASDAHLSIVVVSAASGEGYITVDGNQGDRKNLTLWNNGDKLIETVAENCANTVVVVTSTGQINFEGFADHPNVTAIVWAGPLGDRSGTAIANILFGKANPSGHLPFTIAKTDDDYIPIETYSPSSGEPEDNHLVENDLLVDYRYFEEKNIEPRYAFGYGLSYNEYEVSNAKVSAAKKVDEELPEPATYLSEFSYQNAKDSKNPSDAFAPADLNRVNEYLYPYLDSNVTLKDGNYEYPDGYSTEQRTTPNQPGGGLGGNDALWEVAYNSTDKFVPQGNSTDKFVPQLYLKHPEDGKFETPIQLRGFEKVELSPGEKKTVDLRLLRRDLSVWDTTRQSWIVESGTYEALIGVAVNDIKTSVLFTI

SEQ ID NO: 37 - protein, 488 aa, Trichoderma reesei
MNIFYIFLFLLSFVQGSLNCTLRDSQQKSLVMSGPYELKASLDKREAEAEAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 38 - protein, 547 aa, Trichoderma reesei
MNIFYIFLFLLSFVQGSLNCTLRDSQQKSLVMSGPYELKASLDKREAEAEAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 39 - protein, 492 aa, Trichoderma reesei
MVSFTSLLAGVAAISGVLAAPAAEVEPVAVEKREAEAEAVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 40 - protein, 879 aa, Clostridium thermocellum
MRLVNSLGRRKILLILAVIVAFSTVLLFAKLWGRKTSSTLDEVGSKTHGDLTAENKNGGYLPEEEIPDQPPATGAFNYGEALQKAIFFYECQRSGKLDPSTLRLNWRGDSGLDDGKDAGIDLTGGWYDAGDHVKFNLPMSYSAAMLGWAVYEYEDAFKQSGQYNHILNNIKWACDYFIKCHPEKDVYYYQVGDGHADHAWWGPAEVMPMERPSYKVDRSSPGSTVVAETSAALAIASIIFKKVDGEYSKECLKHAKELFEFADTTKSDDGYTAANGFYNSWSGFYDELSWAAVWLYLATNDSSYLDKAESYSDKWGYEPQTNIPKYKWAQCWDDVTYGTYLLLARIKNDNGKYKEAIERHLDWWTTGYNGERITYTPKGLAWLDQWGSLRYATTTAFLACVYSDWENGDKEKAKTYLEFARSQADYALGSTGRSFVVGFGENPPKRPHHRTAHGSWADSQMEPPEHRHVLYGALVGGPDSTDNYTDDISNYTCNEVACDYNAGFVGLLAKMYKLYGGSPDPKFNGIEEVPEDEIFVEAGVNASGNNFIEIKAIVNNKSGWPARVCENLSFRYFINIEEIVNAGKSASDLQVSSSYNQGAKLSDVKHYKDNIYYVEVDLSGTKIYPGGQSAYKKEVQFRISAPEGTVFNPENDYSYQGLSAGTVVKSEYIPVYDAGVLVFGREPGSASKSTSKDNGLSKATPTVKTESQPTAKHTQNPASDFKTPANQNSVKKDQGIKGEVVLQYANGNAGATSNSINPRFKIINNGTKAINLSDVKIRYYYTKEGGASQNFWCDWSSAGNSNVTGNFFNLSSPKEGADTCLEVGFGSGAGTLDPGGSVEVQIRFSKEDWSNYNQSNDYSFKQACLRQRTLIYLYATWLR

SEQ ID NO: 41 - protein, 625 aa, Arabidopsis thaliana
MGSRTTISILVVLLLGLVQLAISGHDYKQALSKSILFFEAQRSGHLPPNQRVSWRSHSGLYDGKSSGVDLVGGYYDAGDNVKFGLPMAFTVTTMCWSIIEYGGQLESNGELGHAIDAVKWGTDYFIKAHPEPNVLYGEVGDGKSDHYCWQRPEEMTTDRRAYKIDRNNPGSDLAGETAAAMAAASIVFRRSDPSYSAELLRHAHQLFEFADKYRGKYDSSITVAQKYYRSVSGYNDELLWAAAWLYQATNDKYYLDYLGKNGDSMGGTGWSMTEFGWDVKYAGVQTLVAKVLMQGKGGEHTAVFERYQQKAEQFMCSLLGKSTKNIKKTPGGLIFRQSWNNMQFVTSASFLATVYSDYLSYSKRDLLCSQGNISPSQLLEFSKSQVDYILGDNPRATSYMVGYGENYPRQVHHRGSSIVSFNVDQKFVTCRGGYATWFSRKGSDPNVLTGALVGGPDAYDNFADQRDNYEQTEPATYNNAPLLGVLARLISGSTGFDQLLPGVSPTPSPVIIKPAPVPQRKPTKPPASSPSPITISQKMTNSWKNEGKVYYRYSTILTNRSTKTLKILKISITKLYGPIWGVTKTGNSFSFPSWMQSLPSGKSMEFVYIHSASPADVLVSNYSLE

SEQ ID NO: 42 - protein, 237 aa, Aspergillus aculeauts
MKAFHLLAALAGAAVAQQAQLCDQYATYTGGVYTINNNLWGKDAGSGSQCTTVNSASSAGTSWSTKWNWSGGENSVKSYANSGLTFNKKLVSQISQIPTTARWSYDNTGIRADVAYDLFTAADINHVTWSGDYELMIWLARYGGVQPIGSQIATATVDGQTWELWYGANGSQKTYSFVAPTPITSFQGDVNDFFKYLTQNHGFPASSQYLITLQFGTEPFTGGPATLSVSNWSASVQ

SEQ ID NO: 43 - protein, 895 aa, Clostridium thermocellum
MNFRRMLCAAIVLTIVLSIMLPSTVFALEDKSPKLPDYKNDLLYERTFDEGLCFPWHTCEDSGGKCDFAVVDVPGEPGNKAFRLTVIDKGQNKWSVQMRHRGITLEQGHTYTVRFTIWSDKSCRVYAKIGQMGEPYTEYWNNNWNPFNLTPGQKLTVEQNFTMNYPTDDTCEFTFHLGGELAAGTPYYVYLDDVSLYDPRFVKPVEYVLPQPDVRVNQVGYLPFAKKYATVVSSSTSPLKWQLLNSANQVVLEGNTIPKGLDKDSQDYVHWIDFSNFKTEGKGYYFKLPTVNSDTNYSHPFDISADIYSKMKFDALAFFYHKRSGIPIEMPYAGGEQWTRPAGHIGVAPNKGDTNVPTWPQDDEYAGRPQKYYTKDVTGGWYDAGDHGKYVVNGGIAVWTLMNMYERAKIRGIANQGAYKDGGMNIPERNNGYPDILDEARWEIEFFKKMQVTEKEDPSIAGMVHHKIHDFRWTALGMLPHEDPQPRYLRPVSTAATLNFAATLAQSARLWKDYDPTFAADCLEKAEIAWQAALKHPDIYAEYTPGSGGPGGGPYNDDYVGDEFYWAACELYVTTGKDEYKNYLMNSPHYLEMPAKMGENGGANGEDNGLWGCFTWGTTQGLGTITLALVENGLPSADIQKARNNIAKAADKWLENIEEQGYRLPIKQAEDERGGYPWGSNSFILNQMIVMGYAYDFTGNSKYLDGMQDGMSYLLGRNGLDQSYVTGYGERPLQNPHDRFWTPQTSKKFPAPPPGIIAGGPNSRFEDPTITAAVKKDTPPQKCYIDHTDSWSTNEITINWNAPFAWVTAYLDEIDLITPPGGVDPEEPEVIYGDCNGDGKVNSTDAVALKRYILRSGISINTDNADVNADGRVNSTDLAILKRYILKEIDVLPHK

SEQ ID NO: 44 - protein, 438 aa, Agaricus bisporus
MFKFAALLALASLVPGFVQAQSPVWGQCGGNGWTGPTTCASGSTCVKQNDFYSQCLPNNQAPPSTTTQPGTTPPATTTSGGTGPTSGAGNPYTGKTVWLSPFYADEVAQAAADISNPSLATKAASVAKIPTFVWFDTVAKVPDLGGYLADARSKNQLVQIVVYDLPDRDCAALASNGEFSLANDGLNKYKNYVDQIAAQIKQFPDVSVVAVIEPDSLANLVTNLNVQKCANAQSAYKEGVIYAVQKLNAVGVTMYIDAGHAGWLGWPANLSPAAQLFAQIYRDAGSPRNLRGIATNVANFNALRASSPDPITQGNSNYDEIHYIEALAPMLSNAGFPAHFIVDQGRSGVQNIRDQWGDWCNVKGAGFGQRPTTNTGSSLIDAIVWVKPGGECDGTSDNSSPRFDSHCSLSDAHQPAPEAGTWFQAYFETLVANANPAL

SEQ ID NO: 45 - protein, 516 aa, Phanerochaete chrysosporium
MFRTATLLAFTMAAMVFGQQVGTNTAENHRTLTSQKCTKSGGCSNLNTKIVLDANWRWLHSTSGYTNCYTGNQWDATLCPDGKTCAANCALDGADYTGTYGITASGSSLKLQFVTGSNVGSRVYLMADDTHYQMFQLLNQEFTFDVDMSNLPCGLNGALYLSAMDADGGMAKYPTNKAGAKYGTGYCDSQCPRDIKFINGEANVEGWNATSANAGTGNYGTCCTEMDIWEANNDAAAYTPHPCTTNAQTRCSGSDCTRDTGLCDADGCDFNSFRMGDQTFLGKGLTVDTSKPFTVVTQFITNDGTSAGTLTEIRRLYVQNGKVIQNSSVKIPGIDPVNSITDNFCSQQKTAFGDTNYFAQHGGLKQVGEALRTGMVLALSIWDDYAANMLWLDSNYPTNKDPSTPGVARGTCATTSGVPAQIEAQSPNAYVVFSNIKFGDLNTTYTGTVSSSSVSSSHSSTSTSSSHSSSSTPPTQPTGVTVPQWGQCGGIGYTGSTTCASPYTCHVLNPYYSQCY

SEQ ID NO: 46 - protein, 444 aa, Thermotoga neapolitana
MKKFPEGFLWGVATASYQIEGSPLADGAGMSIWHTFSHTPGNVKNGDTGDVACDHYNRWKEDIEIIEKIGAKAYRFSISWPRILPEGTGKVNQKGLDFYNRIIDTLLEKNITPFITIYHWDLPFSLQLKGGWANRDIADWFAEYSRVLFENFGDRVKHWITLNEPWVVAIVGHLYGVHAPGMKDIYVAFHTVHNLLRAHAKSVKVFRETVKDGKIGIVFNNGYFEPASEREEDIRAARFMHQFNNYPLFLNPIYRGEYPDLVLEFAREYLPRNYEDDMEEIKQEIDFVGLNYYSGHMVKYDPNSPARVSFVERNLPKTAMGWEIVPEGIYWILKGVKEEYNPQEVYITENGAAFDDVVSEGGKVHDQNRIDYLRAHIEQVWRAIQDGVPLKGYFVWSLLDNFEWAEGYSKRFGIVYVDYNTQKRIIKDSGYWYSNGIKNNGLTD

SEQ ID NO: 47 - protein, 455 aa, Caldocellum saccharolyticum
MDMSFPKGFLWGAATASYQIEGAWNEDGKGESIWDRFTHQKRNILYGHNGDVACDHYHRFEEDVSLMKELGLKAYRFSIAWTRIFPDGFGTVNQKGLEFYDRLINKLVENGIEPVVTLYHWDLPQKLQDIGGWANPEIVNYYFDYAMLVINRYKDKVKKWITFNEPYCIAFLGYFHGIHAPGIKDFKVAMDVVHSLMLSHFKVVKAVKENNIDVEVGITLNLTPVYLQTERLGYKVSEIEREMVSLSSQLDNQLFLDPVLKGSYPQKLLDYLVQKDLLDSQKALSMQQEVKENFIFPDFLGINYYTRAVRLYDENSSWIFPIRWEHPAGEYTEMGWEVFPQGLFDLLIWIKESYPQIPIYITENGAAYNDIVTEDGKVHDSKRIEYLKQHFEAARKAIENGVDLRGYFVWSLMDNFEWAMGYTKRFGIIYVDYETQKRIKKDSFYFYQQYIKENS

(19) United States
(12) Patent Application Publication
(10) Pub. No.: US 2010/0075363 A1
(43) Pub. Date: Mar. 25, 2010

(54) Recombinant Yeast Strains Expressing Tethered Cellulase Enzymes

(75) Inventors: John E.E. McBride, Hanover, NH (US); Kristen M. Delault, Caanan, NH (US); Lee R. Lynd, Meriden, NH (US); Jack T. Pronk, Schipluiden (NL)

Correspondence Address: LATHROP & GAGE LLP, 4845 PEARL EAST CIRCLE, SUITE 201, BOULDER, CO, 80301, US

(73) Assignee: THE TRUSTEES OF DARTMOUTH COLLEGE, Hanover, NH (US)

(21) Appl. No.: 12/516,175

(22) PCT Filed: Nov. 21, 2007
(86) PCT No.: PCT/US07/85390

§ 371 Date: Jun. 26, 2009

Related U.S. Application Data
(60) Provisional application No. 60/867,018, filed on Nov. 22, 2006.

(51) Int. Cl. C12Q 1/02; C12N 1/19; C12P 7/10; C07H 21/04; C12N 15/74

(52) U.S. Cl. 435/29; 435/254.2; 435/254.21; 435/254.22; 435/254.23; 435/165; 536/23.1; 435/320.1

(57) ABSTRACT

Recombinant yeast strains that saccharify, ferment and grow on insoluble and crystalline forms of cellulose are disclosed herein. The yeast strains express tethered cellulases including cellobiohydrolase, endoglucanase and β-glucosidase. The recombinant organisms are particularly suited for consolidated bioprocessing.

### RELATED APPLICATIONS

This application claims the benefit of priority to U.S. Provisional Patent Application Ser. No. 60/867,018, filed Nov. 22, 2006, which is hereby incorporated by reference in its entirety.

### GOVERNMENT RIGHTS

The U.S. government has certain rights in this invention as provided for by the terms of Grant No. 60NANB1D0064, awarded by the National Institute of Standards and Technology.

### BACKGROUND

1. Field of the Invention

The present invention pertains to the field of biomass processing to produce ethanol and other products. In particular, recombinant organisms that hydrolyze, ferment and grow on soluble and insoluble cellulose are disclosed, as well as methods for the production and use of the organisms.

2. Description of the Related Art

Biomass represents an inexpensive and readily available cellulosic feedstock from which sugars may be produced. These sugars may be recovered or fermented to produce alcohols and/or other products. Among bioconversion products, interest in ethanol is high because it may be used as a renewable domestic fuel.

Significant research has been performed in the areas of reactor design, pretreatment protocols and separation technologies, so that bioconversion processes are becoming economically competitive with petroleum fuel technologies. Further, it has been observed that large cost savings may be obtained when two or more process steps are combined. For example, simultaneous saccharification and fermentation (SSF) and simultaneous saccharification and co-fermentation (SSCF) processes combine enzymatically-mediated saccharification with fermentation in a single reactor or continuous process apparatus.

In addition to savings associated with shorter reaction times and reduced capital costs, co-fermentation processes may also provide improved product yields because certain compounds that would otherwise accrue at levels that inhibit metabolysis or hydrolysis are consumed by the co-fermenting organisms. In one such example, β-glucosidase ceases to hydrolyze cellobiose in the presence of glucose and, in turn, the build-up of cellobiose impedes cellulose degradation. An SSCF process involving co-fermentation of cellulose and hemicellulose hydrolysis products may alleviate this problem by converting the glucose into one or more products that do not inhibit the hydrolytic activity of β-glucosidase.

The ultimate combination of biomass processing steps is referred to as consolidated bioprocessing (CBP). CBP involves four biologically-mediated events: (1) enzyme production, (2) substrate hydrolysis, (3) hexose fermentation and (4) pentose fermentation. These events may be performed in a single step by a microorganism that degrades and utilizes both cellulose and hemicellulose. Development of CBP organisms could potentially result in very large cost reductions as compared to the more conventional approach of producing saccharolytic enzymes in a dedicated process step. CBP processes that utilize more than one organism to accomplish the four biologically-mediated events are referred to as consolidated bioprocessing co-culture fermentations.

### Consolidated Bioprocessing Organisms

Numerous attempts have been made to create recombinant organisms for CBP. For example, various cellulase genes have been expressed in Saccharomyces cerevisiae with the aim of direct ethanol production from cellulose. While short-lived fermentations have been observed using recombinant organisms, sustainable growth of the organisms on cellulose has not been achieved. This is, at least, partially due to the fact that heterologous cellulase enzymes are usually produced by recombinant organisms in such low concentrations that the amount of saccharified substrate available is unable to sustain growth of the organisms. This concentration deficiency is exacerbated when enzymes are secreted into media, where they are further diluted.

In an attempt to alleviate enzyme concentration deficiencies, yeast strains displaying cell surface proteins have recently been developed. Fujita, Y.; Takahashi, S.; Ueda, M.; Tanaka, A.; Okada, H.; Morikawa, Y.; Kawaguchi, T.; Arai, M.; Fukuda, H.; Kondo, A. “Direct and Efficient Production of Ethanol from Cellulosic Material with a Yeast Strain Displaying Cellulolytic Enzymes” Applied and Environmental Microbiology, 68(1), 5136-5141, (2002) describes an S. cerevisiae strain expressing tethered β-glucosidase I (BGLI) and endoglucanase II (EGII). The strain is able to grow on barley β-glucan, which is a linear, soluble polysaccharide. To date, however, there have been no reports of yeast strains expressing cell-surface tethered enzymes that are able to grow on insoluble cellulose, nor have there been reports of any yeast strains able to grow on crystalline cellulose.

As reported by Fan et al. in PCT/US05/018430, expression of cell-surface tethered enzymes may provide an advantage for cell growth, where saccharified substrate is unable to diffuse away from the cell before being metabolized. Further, a portion of a population of cells expressing tethered enzymes may exhibit enhanced expression of the one or more tethered enzymes relative to the overall population. This portion may exhibit enhanced binding to the substrate and improved growth characteristics. As such, observation of these traits may be a useful criteria for organism selection.

### SUMMARY

The present instrumentalities advance the art and overcome the problems outlined above by providing recombinant yeast strains that express tethered cellulase enzymes and have the ability to saccharify insoluble cellulose. Methods for using the recombinant organisms to produce ethanol are also disclosed.

In an embodiment, a transformed yeast cell expresses a plurality of genes, wherein the genes code for expression of tethered enzymes including endoglucanase, cellobiohydrolase and β-glucosidase.

In an embodiment, a transformed organism includes a yeast that in a native state lacks the ability to saccharify cellulose, wherein the yeast is transformed with heterologous polynucleotides that express a plurality of enzymes that confer upon the yeast the ability to saccharify crystalline cellulose.

In an embodiment, an isolated polynucleotide includes (a) a polynucleotide sequence of SEQ ID NO: 11; (b) a polynucleotide sequence of SEQ ID NO: 12; (c) a polynucleotide sequence of SEQ ID NO: 28; (d) a polynucleotide sequence of SEQ ID NO: 29; and (e) a polynucleotide sequence of SEQ ID NO: 30; or (f) a polynucleotide sequence having at least about 90% sequence identity with the polynucleotide sequences of (a)-(e).

A yeast host according to any of the aforementioned embodiments may be utilized in a method for producing ethanol, which includes producing a transformed yeast host and culturing the transformed yeast host in medium that contains cellulose under suitable conditions for a period sufficient to allow saccharification and fermentation of the cellulose to ethanol.

A yeast host according to any of the aforementioned embodiments may be utilized in a method for selecting a transformed yeast cell with enhanced binding affinity for insoluble cellulose. The method includes producing a transformed yeast host, culturing the transformed yeast host under suitable conditions for a period sufficient to allow growth and replication of the transformed yeast host, exposing a sample of transformed yeast host from the culture to the insoluble cellulose and selecting the sample of transformed yeast host that provides at least a two fold reduction in supernatant optical density relative to a similarly cultured and exposed sample of the native organism.

### BRIEF DESCRIPTION OF THE DRAWINGS

FIG. 1 is a schematic of an exemplary δ-integration vector having two cellulase enzymes and a kanamycin marker.

FIG. 2 shows a comparison of recombinant Y294 and CEN.PK yeast transformed to express β-glucosidase I, endoglucanase I, cellobiohydrolase I and cellobiohydrolase II enzymes and untransformed Y294 and CEN.PK yeast growth on phosphoric acid swollen cellulose (PASC), according to an embodiment.

FIG. 3 shows a comparison of recombinant CEN.PK yeast transformed to express β-glucosidase I, endoglucanase I, cellobiohydrolase I and cellobiohydrolase II enzymes and untransformed CEN.PK yeast growth on bacterial microcrystalline cellulose (BMCC), according to an embodiment.

FIG. 4 shows a comparison of recombinant Y294 yeast transformed to express β-glucosidase I and endoglucanase I enzymes; Y294 yeast transformed to express β-glucosidase I, endoglucanase I, cellobiohydrolase I and cellobiohydrolase II enzymes and untransformed Y294 yeast growth on bacterial microcrystalline cellulose (BMCC), according to an embodiment.

FIG. 5 shows a comparison of recombinant yeast transformed to express β-glucosidase I and endoglucanase I enzymes and untransformed yeast cell binding on cellulose particles, according to an embodiment.

FIG. 6 shows cell concentration and viable cell counts for semi-continuous cultures of transformed and untransformed strains of CEN.PK growing on Avicell as a carbon source, according to an embodiment.

### DETAILED DESCRIPTION

There will now be shown and described methods for engineering and utilizing recombinant yeast in the conversion of biomass to ethanol. The disclosed yeast strains express tethered cellulase enzymes, which impart upon the yeast an ability to grow on insoluble non-crystalline and crystalline forms of cellulose.

As used herein, an organism is in “a native state” if it is has not been genetically engineered or otherwise manipulated by the hand of man in a manner that intentionally alters the genetic and/or phenotypic constitution of the organism. For example, wild-type organisms may be considered to be in a native state.

As used herein, a protein is “tethered” to an organism's cell surface if at least one terminus of the protein is covalently and/or electrostatically bound to the cell membrane, or cell wall. It will be appreciated that a tethered protein may include one or more enzymatic regions that may be joined to one or more other types of regions (e.g., a promoter, a terminator, an anchoring domain, a linker, a signaling region, etc.). While the one or more enzymatic regions may not be directly bound to the cell membrane (e.g., such as when binding occurs via an anchoring domain), this protein may nonetheless be considered a “tethered enzyme” according to the present specification.

Tethering may, for example, be accomplished by incorporation of an anchoring domain into a recombinant protein that is heterologously expressed by a cell, e.g., a fatty acid linkage, glycosyl phosphatidyl inositol anchor or other suitable molecular anchor which may bind the tethered protein to the cell membrane of the host cell. In addition, tethering may be accomplished by prenylation, which is the attachment of a hydrophobic chain to a protein to faciliate interaction between the modified protein and the hydrophobic region of the lipid bilayer.

Although the results reported herein are for Saccharomyces cerevisiae, the methods and materials also apply to other types of yeast including Schizosaccharomyces pombe, Candida albicans, Kluyveromyces lactis, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Kluyveromyces marxianus, Issatchenkia orientalis and Schwanniomyces occidentalis. The disclosed methods and materials are useful generally in the field of engineered yeast.

The disclosed recombinant yeast strains have the potential to contribute significant savings in the lignocellulosic biomass to ethanol conversion. For example, recombinant yeast strains may be suitable for a consolidated bioprocessing co-culture fermentation where they would convert cellulose to ethanol, and hemicellulose would be degraded by a pentose-utilizing organism, such as Saccharomyces cerevisiae RWB218, disclosed by Kuyper, M.; Hartog, M. M. P.; Toirkens, M. J.; Almering, M. J. H.; Winkler, A. A.; van Dijken, J. P.; Pronk, J. T. “Metabolic engineering of a xylose-isomerase-expressing Saccharomyces cerevisiae strain for rapid anaerobic xylose fermentation”, FEMS Yeast Research, 5: 399-409, (2005).

It will be appreciated that suitable lignocellulosic material may be any feedstock that contains soluble or insoluble cellulose, where the insoluble cellulose may be in a crystalline or non-crystalline form. In various embodiments, the lignocellulosic biomass comprises wood, corn, corn stover, sawdust, bark, leaves, agricultural and forestry residues, grasses such as switchgrass, ruminant digestion products, municipal wastes, paper mill effluent, newspaper, cardboard or combinations thereof.

In some embodiments, endoglucanase, cellobiohydrolase and β-glucosidase can be any suitable endoglucanase, cellobiohydrolase and/or β-glucosidase derived from, for example, a fungal or bacterial source.

In certain embodiments, endoglucanase(s) can be an endoglucanase I and/or an endoglucanase II isoform, paralogue or orthologue. In another embodiment, endoglucanase expressed by the host cells can be recombinant endo-1,4-β-glucanase. In some embodiments, endoglucanase is an endoglucanase I from Trichoderma reesei. In another embodiment, endoglucanase is encoded by the polynucleotide sequence of SEQ ID NO: 28.

In certain embodiments, β-glucosidase is derived from Saccharomycopsis fibuligera. In some embodiments, β-glucosidase can be a β-glucosidase I and/or a β-glucosidase II isoform, paralogue or orthologue. In another embodiment, β-glucosidase expressed by the host cells can be recombinant β-glucanase I from a Saccharomycopsis fibuligera source.

In certain embodiments, cellobiohydrolase(s) can be a cellobiohydrolase I and/or a cellobiohydrolase II isoform, paralogue or orthologue. In some embodiments, cellobiohydrolases are cellobiohydrolase I and/or cellobiohydrolase II from Trichoderma reesei. In another embodiment, cellobiohydrolases are encoded by the polynucleotide sequences of SEQ ID NOS: 29 and/or 30.

Cellulase catalytic domain genes that are suitable for use in the disclosed recombinant organisms include, for example, those shown in Table 1. Cellulase genes suitable for incorporation into yeast according to the present instrumentalities (e.g., BGLI, EGI, CBHI, CBHII, Endo-1, EG19, glycoside hydrolase, CeI3AC, gghA and BGLA) may be synthesized or isolated from various organisms. Such cellulase genes, and methods for synthesizing and/or isolating the genes, are known in the art. For example, many cellulase catalytic domains can be located in the online ExPASy database (http://www.expasy.org/) under E.C. # 3.2.1.4 (endo-1,4, beta-D-glucanase), E.C.# 3.2.1.91 (cellulose 1,4-beta-cellobiosidase) and E.C.# 3.2.1.21 (beta-glucosidase) [retrieved Nov. 14, 2007]. Retrieved from the Internet: <URL: www.expasy.org/>.

TABLE 1
Cellulase Catalytic Domains

| | Cellulase Catalytic Domain | | SEQ |
|---|---|---|---|
| Name | Amino Acid Sequence | Originating Organism | ID NO |
| BGLI | mvsftsllagvaaisgvlaapaaevepvavekreaea | Saccharmycopsis | 36 |
| | eamlmivqllvfalglavavpiqnytqspsqrdessq | fibuliga | |
| | wvsphyyptpqggrlqdvwqeayarakaivgqmtive | | |
| | kvnlttgtgwqldpcvgntgsvprfgipnlclqdgpl | | |
| | gvrfadfvtgypsglatgatfnkdlflqrgqalghef | | |
| | nskgvhialgpavgplgvkarggrnfeafgsdpylqg | | |
| | taaaatikglqennvmacvkhgigneqekyrqpddin | | |
| | patnqttkeaisanipdramhalylwpfadsvragvg | | |
| | svmcsynrvnntyacensymmnhllkeelgfqgfvvs | | |
| | dwgaqlsgvysaisgldmsmpgevyggwntgtsfwgq | | |
| | nltkaiynetvpierlddmatrilaalyatnsfpted | | |
| | hlpnfsswttkeygnkyyadntteivkvnynvdpsnd | | |
| | ftedtalkvaeesivllknenntlpispekakrllls | | |
| | giaagpdpigyqcedqsctngalfqgwgsgsvgspky | | |
| | qvtpfeeisylarknkmqfdyiresydlaqvtkvasd | | |
| | ahlsivvvsaasgegyitvdgnqgdrknltlwnngdk | | |
| | lietvaencantvvvvtstgqinfegfadhpnvtaiv | | |
| | wagplgdrsgtaianilfgkanpsghlpftiaktddd | | |
| | yipietyspssgepednhlvendllvdyrygeeknie | | |
| | pryafgyglsyneyevsnakvsaakkvdeelpepaty | | |
| | lsefsyqnakdsknpsdafapadlnrvneylypylds | | |
| | nvtlkdgnyeypdgysteqrttpnqpggglggndalw | | |
| | evaynstdkfvpqgnstdkfvpqlylkhpedgkfetp | | |
| | iqlrgfekvelspgekktvdlrllrrdlsvwdttrqs | | |
| | wivesgtyealigvavndiktsvlfti | | |
| EGI | mnifyiflfllsfvqgslnctlrdsqqkslvmsgpye | Trichoderma reesei | 37 |
| | lkasldkreaeaeaqqpgtstpevhpklttykctksg | | |
| | gcvaqdtsvvldwnyrwmhdanynsctvnggvnttlc | | |
| | pdeatcgkncfiegvdyaasgvttsgssltmnqymps | | |
| | ssggyssvsprlylldsdgeyvmlklngqelsfdvdl | | |
| | salpcgengslylsqmdengganqyntaganygsgyc | | |
| | daqcpvqtwrngtlntshqgfccnemdilegnsrana | | |
| | ltphsctatacdsagcgfnpygsgyksyygpgdtvdt | | |
| | sktftiitqfntdngspsgnlvsitrkyqqngvdips | | |
| | aqpggdtisscpsasaygglatmgkalssgmvlvfsi | | |
| | wndnsqymnwldsgnagpcsstegnpsnilannpnth | | |
| | vvfsnirwgdigsttnstapppppassttfsttrrss | | |
| | ttssspsctqthwgqcggigysgcktctsgttcqysn | | |
| | dyysqcl | | |
| CBHI | mnifyiflfllsfvqgslnctlrdsqqkslvmsgpye | Trichoderma reesei | 38 |
| | lkasldkreaeaeaqsactlqsethppltwqkcssgg | | |
| | tctqqtgsvvidanwrwthatnsstncydgntwsstl | | |
| | cpdnetcaknccldgaayastygvttsgnslsigfvt | | |
| | qsaqknvgarlylmasdttyqeftllgnefsfdvdvs | | |
| | qlpcglngalyfvsmdadggvskyptntagakygtgy | | |
| | cdsqcprdlkfingqanvegwepssnnantgigghgs | | |
| | ccsemdiweansisealtphpcttvgqeicegdgcgg | | |
| | tysdnryggtcdpdgcdwnpyrlgntsfygpgssftl | | |
| | dttkkltvvtqfetsgainryyvqngvtfqqpnaelg | | |
| | sysgnelnddyctaeeaefggssfsdkggltqfkkat | | |
| | sggmvlvmslwddyyanmlwldstyptnetsstpgav | | |
| | rgscstssgvpaqvesqspnakvtfsnikfgpigstg | | |
| | npsggnppggnrgttttrrpatttgsspgptqshygq | | |
| | cggigysgptvcasgttcqvlnpyysqcl | | |
| CBHII | mvsftsllagvaaisgvlaapaaevepvavekreaea | Trichoderma reesei | 39 |
| | eavpleerqacssvwgqcggqnwsgptccasgstcvy | | |
| | sndyysqClpgaassssstraasttsrvspttsrsss | | |
| | atpppgstttrvppvgsgtatysgnpfvgvtpwanay | | |
| | yasevsslaipsltgamataaAavakvpsfmwldtld | | |
| | ktpImeqtladirtanknggnyagqfvvydlpdrdca | | |
| | alasngeysiadggvakyknyidtirqivveYsdirt | | |
| | llviepdslanlvtnlgtpkcanaqsaylecinyavt | | |
| | qlnlpnvamyldaghagwlgwpanqdpaaqlfanvyk | | |
| | nassprAlrglatnvanyngwnitsppsytqgnavyn | | |
| | eklyihaigpllanhgwsnaffitdqgrsgkqptgqq | | |
| | qwgdwcnvigtgfgirpsantgdslldsfvwvkpgge | | |
| | cdgtsdssaprfdshcalpdalqpapqagawfqayfv | | |
| | qlltnanpsfl | | |
| Endo-1 | mrlvnslgrrkillilavivafstvllfaklwgrkts | Clostridium | 40 |
| | stldevgskthgdltaenknggylpeeeipdqppatg | thermocellum | |
| | afnygealqkaiffyecqrsgkldpstlrlnwrgdsg | | |
| | lddgkdagidltggwydagdhvkfnlpmsysaamlgw | | |
| | avyeyedafkqsgqynhilnnikwacdyfikchpekd | | |
| | vyyyqvgdghadhawwgpaevmpmerpsykvdrsspg | | |
| | stvvaetsaalaiasiifkkvdgeyskeclkhakelf | | |
| | efadttksddgytaangfynswsgfydelswaavwly | | |
| | latndssyldkaesysdkwgyepqtnipkykwaqcwd | | |
| | dvtygtylllarikndngkykeaierhldwwttgyng | | |
| | eritytpkglawldqwgslryatttafacvysdweng | | |
| | dkekaktylefarsqadyalgstgrsfvvgfgenppk | | |
| | rphhrtahgswadsqmeppehrhvlygalvggpdstd | | |
| | nytddisnytcnevacdynagfvgllakmyklyggsp | | |
| | dpkfngieevpedeifveagvnasgnnfieikaivnn | | |
| | ksgwparvcenlsfryfinieeivnagksasdlqvss | | |
| | synqgaklsdvkhykdniyyvevdlsgtkiypggqsa | | |
| | ykkevqfrisapegtvfnpendysyqglsagtvvkse | | |
| | yipvydagvlvfgrepgsaskstskdnglskatptvk | | |
| | tesqptakhtqnpasdfktpanqnsvkkdqgikgevv | | |
| | lqyangnagatsnsinprfkiinngtkainlsdvkir | | |
| | yyytkeggasqnfwcdwssagnsnvtgnffnlsspke | | |
| | gadtclevgfgsgagtldpggsvevqirfskedwsny | | |
| | nqsndysfkqaclrqrtliylyatwlr | | |
| EG19 | mgsrttisilvvlllglvqlaisghdykqalsksilf | Arabidopsis | 41 |
| | feaqrsghlppnqrvswrshsglydgkssgvdlvggy | thaliana | |
| | ydagdnvkfglpmaftvttmcwsiieyggqlesngel | | |
| | ghaidavkwgtdyfikahpepnvlygevgdgksdhyc | | |
| | wqrpeemttdrraykidrnnpgsdlagetaaamaaas | | |
| | ivfrrsdpsysaellrhahqlfefadkyrgkydssit | | |
| | vaqkyyrsvsgyndellwaaawlyqatndkyyldylg | | |
| | kngdsmggtgwsmtefgwdvkyagvqtlvakvlmqgk | | |
| | ggehtavferyqqkaeqfmcsllgkstknikktpggl | | |
| | ifrqswnnmqfvtsasflatvysdylsyskrdllcsq | | |
| | gnispsqllefsksqvdyilgdnpratsymvgygeny | | |
| | prqvhhrgssivsfnvdqkfvtcrggyatwfsrkgsd | | |
| | pnvltgalvggpdaydnfadqrdnyeqtepatynnap | | |
| | llgvlarlisgstgfdqllpgvsptpspviikpapvp | | |
| | qrkptkppasspspitisqkmtnswknegkvyyryst | | |
| | iltnrstktlkilkisitklygpiwgvtktgnsfsfp | | |
| | swmqslpsgksmefvyihsaspadvlvsnysle | | |
| EGI | mkafhllaalagaavaqqaqlcdqyatytggvytinn | Aspergillus | 42 |
| | nlwgkdagsgsqcttvnsassagtswstkwnwsggen | aculeauts | |
| | svksyansgltfnkklvsqisqipttarwsydntgir | | |
| | advaydlftaadinhvtwsgdyelmiwlaryggvqpi | | |
| | gsqiatatvdgqtwelwygangsqktysfvaptpits | | |
| | fqgdvndffkyltqnhgfpassqylitlqfgtepftg | | |
| | gpatlsvsnwsasvq | | |
| Glycoside | mnfrrmlcaaivltivlsimlpstvfaledkspklpd | Clostridium | 43 |
| hydrolase | ykndllyertfdeglcfpwhtcedsggkcdfavvdvp | thermocellum | |
| | gepgnkafrltvidkgqnkwsvqmrhrgitleqghty | | |
| | tvrftiwsdkscrvyakigqmgepyteywnnnwnpfn | | |
| | ltpgqkltveqnftmnyptddtceftfhlggelaagt | | |
| | pyyvylddvslydprfvkpveyvlpqpdvrvnqvgyl | | |
| | pfakkyatvvssstsplkwqllnsanqvvlegntipk | | |
| | gldkdsqdyvhwidfsnfktegkgyyfklptvnsdtn | | |
| | yshpfdisadiyskmkfdalaffyhkrsgipiempya | | |
| | ggeqwtrpaghigvapnkgdtnvptwpqddeyagrpq | | |
| | kyytkdvtggwydagdhgkyvvnggiavwtlmnmyer | | |
| | akirgianqgaykdggmnipernngypdildearwei | | |
| | effkkmqvtekedpsiagmvhhkihdfrwtalgmlph | | |
| | edpqprylrpvstaatlnfaatlaqsarlwkdydptf | | |
| | aadclekaeiawqaalkhpdiyaeytpgsggpgggpy | | |
| | nddyvgdefywaacelyvttgkdeyknylmnsphyle | | |
| | mpakmgenggangednglwgcftwgttqglgtitlal | | |
| | venglpsadiqkarnniakaadkwlenieeqgyrlpi | | |
| | kqaederggypwgsnsfilqmivmgyaydftgnskyl | | |
| | dgmqdgmsyllgrngldqsyvtgygerplqnphdrfw | | |
| | tpqtskkfpapppgiiaggpnsrfedptitaavkkdt | | |
| | ppqkcyidhtdswstneitinwnapfawvtayldeid | | |
| | litppggvdpeepeviygdcngdgkvnstdavalkry | | |
| | ilrsgisintdnadvnadgrvnstdlailkryilkei | | |
| | dvlphk | | |
| Cel3AC | mfkfaallalaslvpgfvqaqspvwgqcggngwtgpt | Agaricus bisporus | 44 |
| | tcasgstcvkqndfysqclpnnqappstttqpgttpp | | |
| | atttsggtgptsgagnpytgktvwlspfyadevaqaa | | |
| | adisnpslatkaasvakiptfvwfdtvakvpdlggyl | | |
| | adarsknqlvqivvydlpdrdcaalasngefslandg | | |
| | lnkyknyvdqiaaqikqfpdvsvvaviepdslanlvt | | |
| | nlnvqkcanaqsaykegviyavqklnavgvtmyidag | | |
| | hagwlgwpanlspaaqlfaqiyrdagsprnlrgiatn | | |
| | vanfnalrasspdpitqgnsnydeihyiealapmlsn | | |
| | agfpahfivdqgrsgvqnirdqwgdwcnvkgagfgqr | | |
| | pttntgsslidaivwvkpggecdgtsdnssprfdshc | | |
| | slsdahqpapeagtwfqayfetlvananpal | | |
| CBHI | mfrtatllaftmaamvfgqqvgtntaenhrtltsqkc | Phanerochaete | 45 |
| | tksggscnlntkivldanwrwlhstsgytncytgnqw | chrysosporium | |
| | datlcpdgktcaancaldgadytgtygitasgsslkl | | |
| | qfvtgsnvgsrvylmaddthyqmfqllnqeftfdvdm | | |
| | snlpcglngalylsamdadggmakyptnkagakygtg | | |
| | ycdsqcprdikfingeanvegwnatsanagtgnygtc | | |
| | ctemdiweanndaaaytphpcttnaqtrcsgsdctrd | | |
| | tglcdadgcdfnsfrmgdqtflgkgltvdtskpftvv | | |
| | tqfitndgtsagtlteirrlyvqngkviqnssvkipg | | |
| | idpvnsitdnfcsqqktafgdtnfyaqhgglkqvgea | | |
| | lrtgmvlalsiwddyaanmlwldsnyptnkdpstpgv | | |
| | argtcattsgvpaqieaqspnayvvfsnikfgdlntt | | |
| | ytgtvssssvssshsststssshsssstpptqptgvt | | |
| | vpqwgqcggigytgsttcaspytchvlnpyysqcy | | |
| gghA | mkkfpegflwgvatasyqiegspladgagmsiwhtsh | Thermotoga | 46 |
| | tpgnvkngdtgdvacdhynrwkedieiiekigakayr | neapolitana | |
| | fsiswprilpegtgkvnqkgldfynriidtlleknit | | |
| | pfitiyhwdlpfslqlkggwanrdiadwfaeysrvlf | | |
| | enfgdrvkhwitlnepwvvaivghlygvhapgmkdiy | | |
| | vafhtvhnllrahaksvkvfretvkdgkigivfnngy | | |
| | fepasereediraarfmhqfnnyplflnpiyrgeypd | | |
| | lvlefareylprnyeddmeeikqeidfvglnyysghm | | |
| | vkydpnsparvsvernlpktamgweivpegiywilkg | | |
| | vkeeynpqevyitengaafddvvseggkvhdqnridy | | |
| | lrahieqvwraiqdgvplkgyfvwslldnfewaegys | | |
| | krfgivyvdyntqkriikdsgywysngiknngltd | | |
| BGLA | mdmsfpkgflwgaatasyqiegawnedgkgesiwdrf | Caldocellum | 47 |
| | thqkrnilyghngdvacdhyhrfeedvslmkelglka | saccharolyticum | |
| | yrfsiawtrifpdgfgtvnqkglefydrlinklveng | | |
| | iepvvtlyhwdlpqklqdiggwanpeivnyyfdyaml | | |
| | vinrykdkvkkwitfnepyciaflgyfhgihapgikd | | |
| | fkvamdvvhslmlshfkvvkavkennidvevgitlnl | | |
| | tpvylqterlgykvseieremvslssqldnqlfldpv | | |
| | lkgsypqklldylvqkdlldsqkalsmqqevkenfif | | |
| | pdflginyytravrlydensswifpirwehpageyte | | |
| | mgwevfpqglfdlliwikesypqipiyitengaaynd | | |
| | ivtedgkvhdskrieylkqhfeaarkaiengvdlrgy | | |
| | fvwslmdnfewamgytkrfgiiyvdyetqkrikkdsf | | |
| | yfyqqyikens | | |

### EXAMPLES

### Materials

Strain Y294 was obtained from Dr. W. H. Emile van Zyl, University of Stellenbosch, South Africa. BGLI from Saccharomycopsis fibuligera was derived from a plasmid supplied by Dr. van Zyl. CEN.PK 113-11C was obtained from Dr. Peter Koller, Universitat Frankfurt, Germany. The KanMX4 marker used in the integrating vector was derived by PCR from Plasmid M4297 provided by Dr. David Stillman, The University of Utah, U.S.A. The zeocin marker was derived by PCR from the vector pTEF1-Zeo, purchased from Invitrogen, Carlsbad, Calif.

### Media and Strain Cultivation

Escherichia coli strain DH5α (Invitrogen) was used for plasmid transformation and propagation. Cells were grown in LB medium (5 g/L yeast extract, 5 g/L NaCl, 10 g/L tryptone) supplemented with ampicillin (100 mg/L), kanamycin (50 mg/L) or zeocin (20 mg/L). When zeocin selection was desired LB was adjusted to pH 7.0. Fifteen grams per liter agar was added when solid media was desired.

Saccharomyces cerevisiae strains—Y294 (alpha leu2-3,112 ura3-52 his3 trp1-289); BJ5464 (MATalpha ura3-52 trp1 leu2-delta1 his3-delta200 pep4::HIS3 prbl-delta1.6R can1 GAL) and CEN.PK 113-11C (MATa, ura3-52, his3-delta1)—were grown in YPD (10 g/L yeast extract, 20 g/L peptone, 20 g/L glucose) or YPC (10 g/L yeast extract, 20 g/L peptone, 20 g/L cellobiose) media with either G418 (250 mg/L unless specified) or zeocin (20 mg/L unless specified) for selection. Fifteen grams per liter agar was added for solid media.

### Example 1

### Methods for Engineering Saccharomyces cerevisiae Strains with Tethered Cellulase Enzymes

### Molecular Methods

Standard protocols were followed for DNA manipulations (Sambrook, J.; Fritsch, E.; Maniatis, T. Molecular cloning: A laboratory manual. New York: Cold Spring Harbor Laboratory Press; 1989). PCR was performed using Phusion Polymerase (New England Biolabs, Ipswich, Mass.) for cloning, and Taq polymerase (New England Biolabs) for screening transformants. Manufacturer's guidelines were followed as supplied. Restriction enzymes were purchased from New England Biolabs and digests were set up according to the supplied guidelines. Ligations were performed using the Quick Ligation Kit (New England Biolabs) as specified by the manufacturer. Gel purification was performed using either Qiagen or Zymo research kits, PCR product and digest purifications were performed using Zymo research kits, and Qiagen midi and miniprep kits were used for purification of plasmid DNA. Sequencing was performed by the Molecular Biology Core Facility at Dartmouth College.

### Synthetic DNA Constructs

Sequences for CBHI, CBHII and EGI from Trichoderma reesei, linker proteins, secretion signals, and anchoring domains were codon optimized for expression in Saccharomyces cerevisiae using either software provided by DNA 2.0, Menlo Park, Calif., or using “Synthetic Gene Designer” (Wu, G.; Bashir-Bello, N.; Freeland, S. J. “The Synthetic Gene Designer: A flexible web platform to explore sequence manipulation for heterologous expression” Protein Expr. Purif. 47(2): 441-445, (2006)). The optimized sequences are disclosed as SEQ ID NOS: 27-35.

### Construction of a δ-Integrating Vector

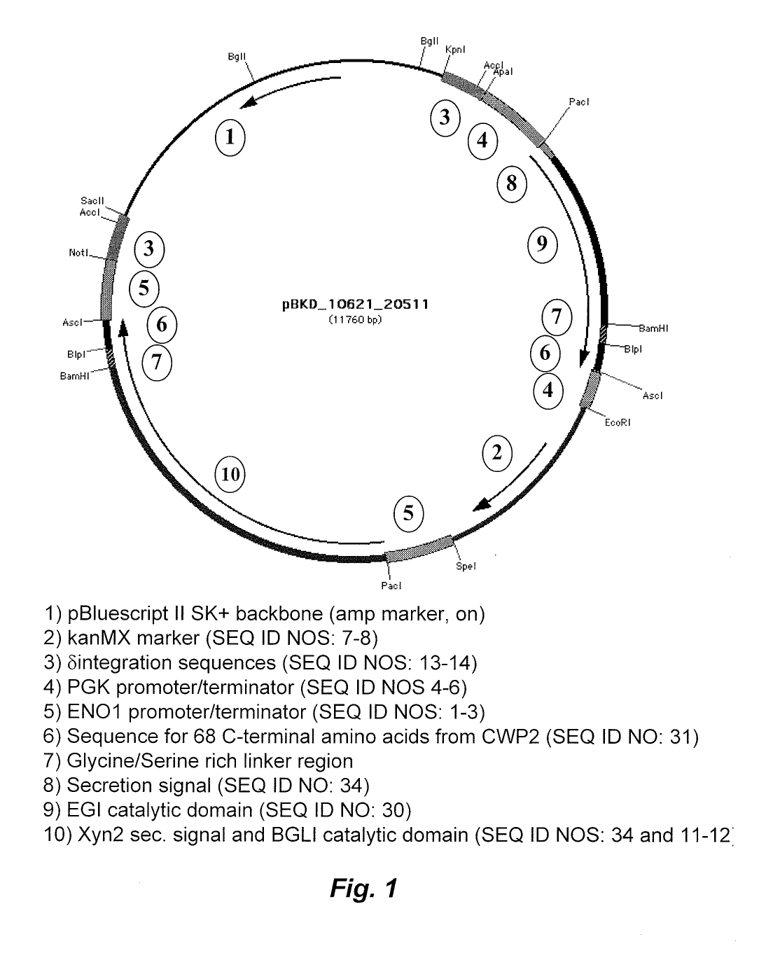

Vectors for integration into the S. cerevisiae genome in multiple copies were made in a number of steps. FIG. 1 shows an example of the final vector including two operons. Each operon includes a cellulase gene (9 or BGLI of 10) linked to a secretion signal (8 or xyn2 of 10), that drives constitutive expression, as well as an anchoring domain (6) that facilitates attachment of the cellulase to the cell membrane. The cellulase gene, secretion signal and anchoring domain are flanked by a set of promoter/terminator sequences (4 or 5). The vector was constructed with two different dominant selectable markers, kanMX and TEF1/zeo. These markers were added to pBluescript II SK+ by first generating PCR fragments (primers SEQ ID NOS: 7 and 8 with plasmid 3, Table 2; SEQ ID NOS: 9 and 10 with plasmid 2, Table 2), digesting the fragments with EcoRI and SpeI, and ligating into the doubly digested (EcoRI/SpeI) pBluscript backbone. The constructs were confirmed first by selecting for E. coli strains resistant to both ampicillin (pBluescript backbone) and either kanamycin or zeocin, as well as by restriction digest to confirm the size of the insert.

TABLE 2
Plasmids

| | | | Reference/ |
|---|---|---|---|
| # | Name of Plasmid | Used for/Genes carried | accession # |
| 1 | pBluescript II SK+ | Expression vector backbone | X52328 |
| | | for assembling expression | |
| | | cassettes | |
| 2 | pTEF1-zeo | TEF1/Zeo marker | Invitrogen |
| 3 | M4297 | KanMX marker | Prof. David |
| | | | Stillman |
| 4 | ySFI | BGLI | Van Rooyen |
| | | | (2005) |
| 5 | pBK | pBluescript; KanMX marker | This work |
| 6 | pBZ | pBluescript; TEF1/Zeo marker | This work |
| 7 | pBK_1 | pBK + PGK P/T* | This work |
| 8 | pBK_2 | pBK + ENO1 P/T* | This work |
| 9 | pBZ_1 | pBZ + PGK P/T* | This work |
| 10 | pBZ_2 | pBZ + ENO1 P/T* | This work |
| 11 | pBKD1_1 | pBK_1 + 1 δ sequence | This work |
| 12 | pBKD1_2 | pBK_2 + 1 δ sequence | This work |
| 13 | pBZD1_1 | pBZ_1 + 1 δ sequence | This work |
| 14 | pBZD1_2 | pBZ_2 + 1 δ sequence | This work |
| 15 | pBKD_1 | pBK_1 + 2 δ sequences | This work |
| 16 | pBZD_1 | pBK_2 + 2 δ sequences | This work |
| 17 | pBKD_2 | pBZ_1 + 2 δ sequences | This work |
| 18 | pBZD_2 | pBZ_2 + 2 δ sequences | This work |
| 19 | pBKD_10001 | pBKD_1 + L1_A1 (original | This work |
| | | optimization) | |
| 20 | pBKD_20001 | pBKD_2 + L1_A1 (original | This work |
| | | optimization) | |
| 21 | pBZD_20001a | pBZD_2 + L2_A1a (re- | This work |
| | | optimized) | |
| 22 | pBKD_20511 | pBKD_20001 + BGL1 | This work |
| 23 | pBKD_11621 | pBKD_10001 + S16 + C2 | This work |
| 24 | pBKD_10621 | pBKD_10001 + S06 + C2 | This work |
| 25 | pBKD_10621_20511 | pBKD_10621 + 20511 (i.e., | This work |
| | | only the cellulase construct) | |
| 26 | pBKD_11621_20511 | pBKD_11621 + 20511 (i.e., | This work |
| | | only the cellulase construct) | |
| 27 | pBZD_11631 | pBZD_1 + S16 + C3_L2_A1 | This work |
| 28 | pBZD_20641 | pBZD_20001a + C4_L3 | This work |
| 29 | pBZD_11631_20641 | pBZD_11631 + 20641 (i.e., | This work |
| | | only the cellulase construct) | |

*P/T = Promoter/Terminator

Promoter/Terminator (P/T) expression regions containing a multiple cloning site were made by overlap PCR using genomic DNA purified from S. cerevisiae strain Y294 and SEQ ID NOS: 1-3 and SEQ ID NOS: 4-6 for the enolase 1 (ENO1) and phosphoglycerate kinase (PGK), respectively. The first round of PCR utilized the forward and overlap primers (SEQ ID NOS: 1-2 or SEQ ID NOS: 4-5), and the second used the product of the first reaction and the reverse primer (SEQ ID NO: 3 or SEQ ID NO: 6). The products of these reactions were further amplified using only the forward (SEQ ID NO: 1 or SEQ ID NO: 4) and reverse primers (SEQ ID NO: 3 or SEQ ID NO: 6). These regions were restriction cloned into both pBK and pBZ using the ApaI and EcoRI sites encoded in the primers and in pBK and pBZ, creating plasmids 7-10, Table 2. The P/T constructs were sequenced using primers SEQ ID NOS: 15 and 16. The sequences matched the expected sequences exactly, with the exception of a few variations from the published PGK terminator sequences.

The sequences for integration at the δ sites in the S. cerevisiae genome were cloned into the backbone as follows. One copy was inserted by digesting SEQ ID NO: 27 from the plasmid supplied by DNA 2.0 with ApaI and KpnI, and ligating the resulting piece with ApaI/KpnI doubly digested plasmids 7-10, creating plasmids 11-14 (Table 2). A second copy was generated by performing PCR with SEQ ID NOS: 13 and 14 on the plasmid from DNA 2.0 containing the 6 region, digesting the resulting fragment and plasmids 11-14 (Table 2) with NotI and SaclI, and performing the ligations. This resulted in plasmids 15-18 (Table 2). The resulting constructs were again sequenced with primers SEQ ID NOS: 15 and 16 to verify the presence of two δ sequences.

An optimized portion of the cell wall gene cwp2 and a flexible linker region between the cellulase and cell wall anchor (cwp2) were then added to the backbone. Plasmids 19 and 20 (Table 2) were constructed by digesting SEQ ID NO: 31 with BamHI and AscI and plasmids 15 and 17 (Table 2) and ligating the resulting fragments. Likewise, plasmid 21 (Table 2) was created by digesting SEQ ID NO: 29 and plasmid 17 with BamHI and AscI and ligating the appropriate fragments.

Cellulase constructs could then be added to the backbone expression vectors in a single, triple ligation step. β-Glucosidase from Saccharomycopsis fibuligera (BGLI) did not require the triple ligation as it already had a secretion signal. Therefore, it was prepared by PCR from plasmid 4 (Table 2) using primers comprising SEQ ID NOS: 11 and 12, digested with PacI and BamHI, and ligated with a PacI/BamHI digested plasmid 20, to create plasmid 22 (Table 2). Plasmids 23 and 24 for synthetic EGI expression were created by digesting SEQ ID NOS: 32 and 34 with MlyI and PacI, SEQ ID NO: 28 with MlyI and BamHI, and plasmid 19 (Table 2) with PacI and BamHI, purifying the appropriate fragments, and ligating all together. Plasmid 27 for CBHI expression was created by digesting SEQ ID NO: 34 with MlyI and PacI, SEQ ID NO: 29 with MlyI and AscI, plasmid 16 with PacI and AscI, and ligating these fragments in a triple ligation. Plasmid 28 was created by triple ligation of MlyI and PacI digested SEQ ID NO: 32, MlyI and BlpI digested SEQ ID NO: 30, and PacI and BlpI digested plasmid 21. These new constructs were sequence verified using primers SEQ ID NOS: 6 and 17 for the EGI and CBHI constructs, and primers SEQ ID NOS: 3 and 18 for the BGL and CBHII constructs.

Constructs for expressing two cellulase constructs simultaneously (either EGI and BGLI or CBHI and CBHII) were constructed by ligating the NotI/SpeI fragment of either plasmid 22 with NotI/SpeI digested plasmids 23 and 24, or by ligating the NotI/SpeI fragment of plasmid 28 with NotI/SpeI digested plasmid 27. These reactions resulted in plasmids 25, 26 and 29, which were sequenced to confirm the presence of both cellulase constructs using primers comprising SEQ ID NOS: 1, 3, 4 and 6.

### Yeast Transformation

A protocol for electrotransformation of yeast was developed based on Cho, K. M.; Yoo, Y. J.; Kang, H. S. “delta-Integration of endo/exo-glucanase and beta-glucosidase genes into the yeast chromosomes for direct conversion of cellulose to ethanol” Enzyme And Microbial Technology, 25: 23-30, (1999) and Ausubel, F. M.; Brent, R.; Kingston, R.; Moore, D.; Seidman, J.; Smith, J.; Struhl, K. Current protocols in molecular biology. USA: John Wiley and Sons, Inc. 1994. Linear fragments of DNA were created by digesting the desired vector with AccI and either BglI (for plasmids 22-26) or FspI (for plasmid 29). AccI has a unique site in the δ sequence and each of the other two enzymes cuts the pBluescript backbone in two places. The fragments were purified by precipitation with 3M sodium acetate and ice cold ethanol, subsequent washing with 70% ethanol, and resuspension in USB dH₂O (DNAse and RNAse free, sterile water) after drying in a 70° C. vacuum oven.

Yeast cells for transformation were prepared by growing to saturation in 5 mL YPD cultures. 4 mL of the culture was sampled, washed 2× with cold distilled water, and resuspended in 640 μL cold distilled water. 80 μL of 100 mM Tris-HCl, 10 mM EDTA, pH 7.5 (10×TE buffer—filter sterilized) and 80 μL of 1M lithium acetate, pH 7.5 (10× liAc—filter sterilized) were added and the cell suspension was incubated at 30° C. for 45 minutes with gentle shaking. 20 μL of 1M DTT was added and incubation continued for 15 minutes. The cells were then centrifuged, washed once with cold distilled water, and once with electroporation buffer (1M sorbitol, 20 mM HEPES), and finally resuspended in 267 μL electroporation buffer.

For electroporation, 10 μg of linearized DNA (measured by estimation on gel) was combined with 50 μL of the cell suspension in a sterile 1.5 mL microcentrifuge tube. The mixture was then transferred to a 0.2 cm electroporation cuvette, and a pulse of 1.4 kV (200Ω, 25 μF) was applied to the sample using the Biorad Gene Pulser device. 1 mL of YPD with 1M sorbitol adjusted to pH 7.0 (YPDS) was placed in the cuvette and the cells were allowed to recover for ˜3 hrs. 100-200 μL cell suspension were spread out on YPDS agar plates with appropriate antibiotic, which were incubated at 30° C. for 3-4 days until colonies appeared. Table 3 contains the genotypes of the yeast strains created.

TABLE 3
Strains of S. cerevisiae created

| | | Contains cellulase constructs |
|---|---|---|
| Name | Starting strain | from this (these) plasmid(s) |
| Y_A1 | Y294 | pBKD_11621_20511 |
| Y_A2 | Y294 | pBKD_10621_20511 |
| Y_A3 | Y294 | pBKD_10421_20511 |
| Y_A4 | Y294 | pBKD_11721_20511 |
| CP1_A1 | CEN.PK 113-11C | pBKD_11621_20511 |
| CP1_A2 | CEN.PK 113-11C | pBKD_10621_20511 |
| CP1_A3 | CEN.PK 113-11C | pBKD_10421_20511 |
| CP1_A4 | CEN.PK 113-11C | pBKD_11721_20511 |
| BJ1_A1 | BJ5464 | pBKD_11621_20511 |
| BJ1_A2 | BJ5464 | pBKD_10621_20511 |
| BJ1_A3 | BJ5464 | pBKD_10421_20511 |
| BJ1_A4 | BJ5464 | pBKD_11721_20511 |
| Y_A1_C1 #1 | Y_A1 | pBKD_11621_20511; |
| | | pBZD_11631_20641 |
| Y_A1_C1 #2 | Y_A1 | pBKD_11621_20511; |
| | | pBZD_11631_20641 |
| Y_A1_C1 #3 | Y_A1 | pBKD_11621_20511; |
| | | pBZD_11631_20641 |
| Y_A1_C1 #5 | Y_A1 | pBKD_11621_20511; |
| | | pBZD_11631_20641 |
| Y_A1_C1 #6 | Y_A1 | pBKD_11621_20511; |
| | | pBZD_11631_20641 |
| CP1_A1_C1 #1 | CP1_A1 | pBKD_11621_20511; |
| | | pBZD_11631_20641 |
| CP1_A1_C1 #6A | CP1_A1 | pBKD_11621_20511; |
| | | pBZD_11631_20641 |
| CP1_A1_C1 #11 | CP1_A1 | pBKD_11621_20511; |
| | | pBZD_11631_20641 |
| CP1_A1_C1 #12 | CP1_A1 | pBKD_11621_20511; |
| | | pBZD_11631_20641 |
| CP1_A1_C1 #17 | CP1_A1 | pBKD_11621_20511; |
| | | pBZD_11631_20641 |
| BJ1_A1_C1 #7 | BJ1_A1 | pBKD_11621_20511; |
| | | pBZD_11631_20641 |
| CP1_A1_C1 #10 | BJ1_A1 | pBKD_11621_20511; |
| | | pBZD_11631_20641 |

### Enzyme Assays

β-Glucosidase activity was measured in a manner similar to that described by McBride, J. E.; Zietsman, J. J.; Van Zyl, W. H.; and Lynd, L. R. “Utilization of cellobiose by recombinant beta-glucosidase-expressing strains of Saccharomyces cerevisiae: characterization and evaluation of the sufficiency of expression” Enzyme And Microbial Technology, 37: 93-101, (2005), except that the volume of the assay was decreased and the reaction performed in a microtiter plate. Briefly, yeast strains were grown to saturation in YPD or YPC media with or without appropriate antibiotics; the optical density at 600 nm (OD(600)) was measured; and a 0.5 mL sample of the culture was centrifuged, the supernatant was separated and saved, and the cell pellet was washed two times with 50 mM citrate buffer, pH 5.0. Reactions for supernatants were made up of 50 μL sample, 50 μL citrate buffer, and 50 μL 20 mM p-nitrophenyl-β-D-glucopyranoside (PNPG) substrate. Reactions with washed cells consisted of 25 μL of cells, 75 μL citrate buffer, and 50 PNPG substrate. If activity was too high for the range of the standard curve, a lower cell concentration was used and the assay was re-run. The standard curve consisted of a 2-fold dilution series of nitrophenol (PNP) standards, starting at 500 nM, and ending at 7.8 nM, and a buffer blank was included. After appropriate dilutions of supernatant or cells were prepared, the microtiter plate was incubated at 37° C. for 10 minutes along with the reaction substrate. The reaction was carried out by adding the substrate, incubating for 30 minutes, and stopping the reaction with 150 μL of 2M Na₂CO₃. The plate was then centrifuged at 2500 rpm for 5 minutes, and 150 μL of supernatant was transferred to another plate. The absorbance at 405 nm was read for each well.

Endoglucanase activity was qualitatively detected by observing clearing zones on synthetic complete media plates (as above, but including 20 g/L glucose) with 0.1% carboxymethyl cellulose (CMC) stained with Congo red (Beguin, P. “Detection of Cellulase Activity in Polyacrylamide Gels using Congo Red-Stained Agar Replicas” Analytical Biochemistry, 131: 333-336, (1983)). Cells were grown for 2-3 days on the plates and were washed off the plate with 1M Tris-HCl buffer, pH 7.5. The plates were then stained for 10 minutes with a 0.1% Congo red solution, and extra dye was subsequently washed off with 1M NaCl.

### Verification of Transformants

For EGI and BGLI transformants, activities were verified by enzyme assay as specified above. For strains where all four cellulases were transformed, PCR with primers SEQ ID NOS: 19-26 was used to verify the presence in genomic DNA of each of the genes being expressed.

After genetic confirmation of the presence of the genes, strains were grown in rich media (YPD) to saturation, and ˜10̂7 cells were washed once with sterile Tris-HCl buffer and inoculated into 10 mL of liquid media in a sealed hungate tube with an air atmosphere. Cell counts were performed on samples taken over time using a haemocytometer. Cell density was measured by spectrophotometry after digestion of the samples with a commercial cellulase preparation (Spezyme CP) added with buffer and sodium azide to inhibit subsequent growth of the cultures. The digestion procedure was verified by plotting the cell number/mL against the OD(600). A value of 3*10̂7 cells/mL=1 OD(600) was obtained.

Growth media with cellulose substrates as the sole carbon source were made using the non-glucose components of synthetic complete medium for yeast including, yeast nitrogen base without amino acids −1.7 g/L, ammonium sulfate −5 g/L, and supplemented with amino acids. Ten milliliters of PASC media (prepared at 2% dry weight) or BMCC media (prepared at 1% dry weight) were placed in sealed hungate tubes for growth experiments.

### Example 2

### Saccharomyces cerevisiae Strains with Tethered Cellulase Enzymes Capable of Growing on Phosphoric Acid Swollen Cellulose (PASC)

Endoglucanase I (EGI), cellobiohydrolase I (CBHI) and cellobiohydrolase II (CBHII) from Trichoderma reesei, along with β-glucosidase I (BGLI) from Saccharomycopsis fibuligera, were expressed as tethered proteins to the Saccharomyces cerevisiae cell surface by fusion with the C-terminal portion of cwp2 from S. cerevisiae, as described above.

For growth experiments on phosphoric acid swollen cellulose (PASC) media, PASC was added as the sole carbon source to synthetic complete medium for yeast at a concentration of 20 g/L. Phosphoric acid swollen cellulose (PASC) was prepared as in Zhang, Y. H.; Cui, J.; Lynd, L. R.; Kuang, L. S. “A transition from cellulose swelling to cellulose dissolution by o-phosphoric acid: evidence from enzymatic hydrolysis and supramolecular structure” Biomacromolecules, 7, 644-648 (2006), with slight modification. Avicel PH105 (10 g) was wetted with 100 mL of distilled water in a 4 L flask. Eight hundred milliliters of 86.2% phosphoric acid was added slowly to the flask with a first addition of 300 mL followed by mixing and subsequent additions of 50 mL aliquots. The transparent solution was kept at 4° C. for 1 hour to allow complete solubilization of the cellulose, at which point no lumps remained in the reaction mixture. Next, 2 L of ice-cooled distilled water were added in 500 mL aliquots with mixing between additions. Three hundred milliliter aliquots of the mixture were centrifuged at 5,000 rpm for 20 minutes at 2° C. and the supernatant removed. Addition of 300 mL cold distilled water and subsequent centrifugation was repeated four times. 4.2 mL of 2M sodium carbonate and 300 mL of water were added to the cellulose, followed by two or three washes with distilled water, until the final pH was ˜6. Samples were dried to constant weight in a 70° C. vacuum oven to measure the dry weight.

Growth experiments carried out in sealed hungate tubes as described above, were sampled by syringe, and the cells were counted. Additionally, samples were digested at 37° C. with a commercial cellulase preparation and sodium azide until all substrate was digested. The absorbance at 600 nm was then taken to measure the cell density. Post digestion OD(600) measurements correlated as expected with cell counts done by haemocytometer.

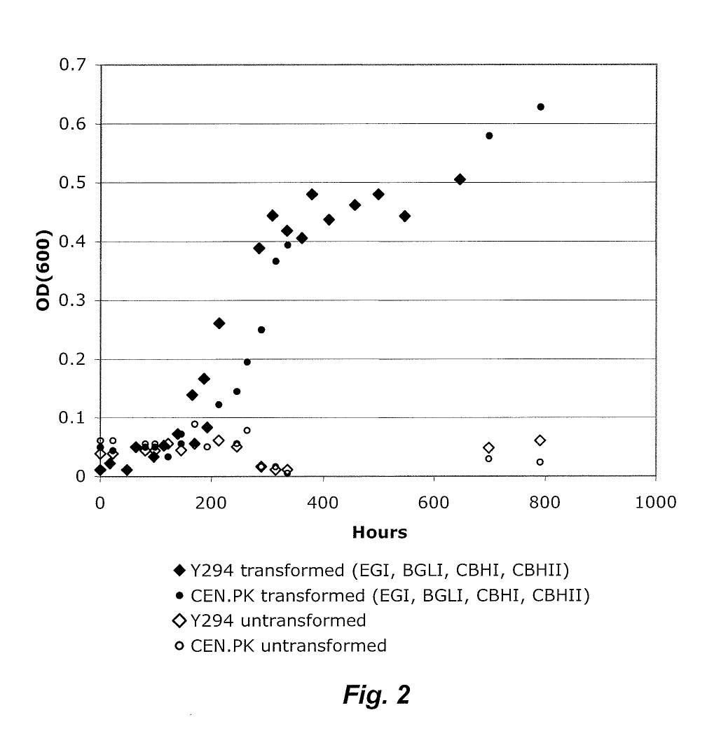

FIG. 2 shows the OD(600) results for growth of native (untransformed) and recombinant strains of Saccharomyces cerevisiae on PASC. Strains created in the Y294 and CEN.PK backgrounds expressing all four cellulase enzymes showed slow, but significant increases in OD(600) over the course of the growth experiment. Untransformed controls from both strains showed no increase in OD(600) over the course of the eight hundred hour growth experiment.

### Example 3

### Saccharomyces cerevisiae Strains with Tethered Cellulase Enzymes Capable of Growing on Bacterial Microcrystalline Cellulose (BMCC)

Endoglucanase I (EGI), cellobiohydrolase I (CBHI) and cellobiohydrolase II (CBHII) from Trichoderma reesei, along with β-glucosidase I (BGLI) from Saccharomycopsis fibuligera, were expressed as tethered proteins to the Saccharomyces cerevisiae cell surface by fusion with the C-terminal portion of cwp2 from S. cerevisiae as described above.

For growth experiments in bacterial microcrystalline cellulose (BMCC) containing media, BMCC was added as the sole carbon source to synthetic complete medium for yeast at a concentration of 10 g/L. Bacterial microcrystalline cellulose (BMCC) was prepared in a similar manner to Jung, H.; Wilson, D. B.; Walker, L. P. “Binding and Reversibility of Thermobifida fusca Cel5A, Cel6B, and Cel48A and their respective catalytic domains to bacterial microcrystalline cellulose” Biotechnology and Bioengineering, 84, 151-159, (2003), except that sodium azide was not added during reconstitution, and washing was carried out by washing and centrifugation five times with distilled water. Quadruplicate 1 mL samples were frozen and then freeze dried to determine the dry weight of the final BMCC suspension.

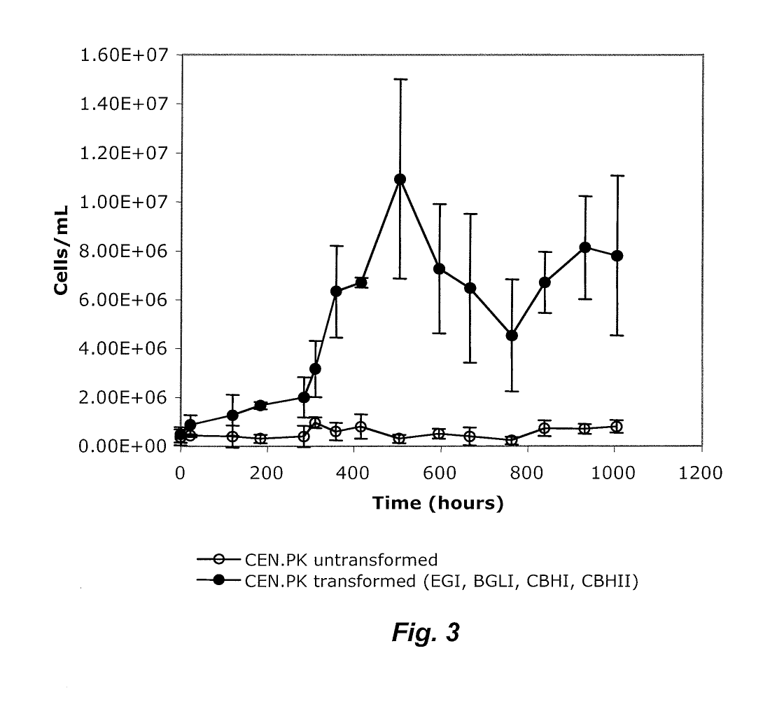

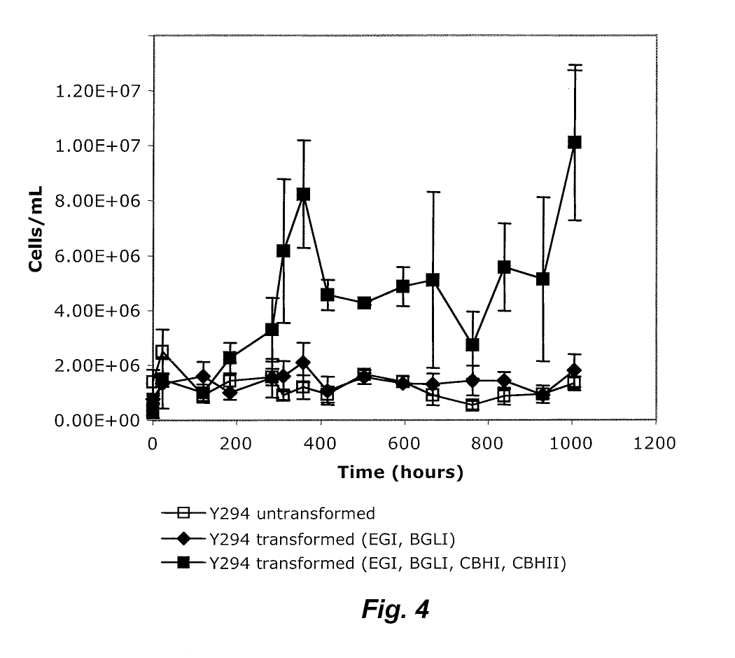

FIGS. 3 and 4 show cell count results for growth of native (untransformed) and recombinant yeast strains of Saccharomyces cerevisiae on BMCC. Strains created in the Y294 and CEN.PK backgrounds expressing all four cellulase enzymes showed a slow, but significant increase in cell counts/mL over the course of the growth experiment. Y294 expressing only BGLI and EGI showed no increase in cell counts/mL over the course of the experiment. Untransformed controls from both strains showed no increase in cell counts over the course of the approximately seven hundred hour growth experiment. These results demonstrate the necessity of utilizing all four cellulases to achieve growth on BMCC when the cellulases are tethered.

### Example 4

### Recombinant Yeast Strains with Enhanced Cellulose Binding Properties

Endoglucanse I (EGI) from Trichoderma reesei and β-glucosidase I (BGLI) from Saccharomycopsis fibuligera were expressed as tethered proteins to the Saccharomyces cerevisiae cell surface by fusion with the C-terminal portion of cwp2 from S. cerevisiae, as described above.

In order to screen the transformed strains for the best cellulose binding individuals, strains expressing tethered enzymes were grown to saturation in 5 mL rich media (˜10̂9 total cells). Fifty, ten, or 0.25 mg of ELCHEMA P100 cellulose was washed 5-8 times with distilled water and autoclaved. The cellulose was then added to each enzyme preparation and allowed to settle to the bottom of the tube. The cell containing supernatant was then removed, and the cellulose pellet was resuspended in sterile 50 mM Tris-HCl buffer, pH 7.5. The pellet was allowed to settle again and the buffer was removed. This process was repeated four more times before rich media was added back to the tube containing the cellulose pellet and cells were allowed to grow again to saturation. The selection procedure was performed a number of times for both transformed strains expressing the cellulase enzymes and the untransformed strains.

A cellulose binding assay was used to examine the original and selected strains. The assay was adapted from Ito, J.; Fujita, Y.; Ueda, M.; Fukuda, H.; Kondo, A. “Improvement of cellulose-degrading ability of a yeast strain displaying Trichoderma reesei endoglucanase II by recombination of cellulose-binding domains” Biotechnology Progress, 20: 688-691, (2004) and Nam, J.; Fujita, Y.; Arai, T.; Kondo, A.; Morikawa, Y.; Okada, H.; Ueda, M.; Tanka, A. “Construction of engineered yeast with the ability of binding to cellulose” Journal of Molecular Catalysis B: Enzymatic 17: 197-202, (2002). Cells from a saturated culture grown in rich media were washed twice in citrate buffer, pH 5.0. They were resuspended in citrate buffer at an OD(600)=2.0, or ˜6*10̂7 cells/mL, in a volume of 2.75 mL and allowed to sit upright in a test tube for ten minutes. A 0.25 mL sample was taken to measure the initial OD(600) of the suspension. A half milliliter of a 10% solution of cellulose (Avicel PH101) was added to each tube. The tubes were then mixed at room temperature and allowed to stand upright for ten minutes. (Ito and Nam used incubations at 4° C. for 24 hours before standing the tubes upright.) A second 0.25 mL sample was obtained and the OD(600) measured.

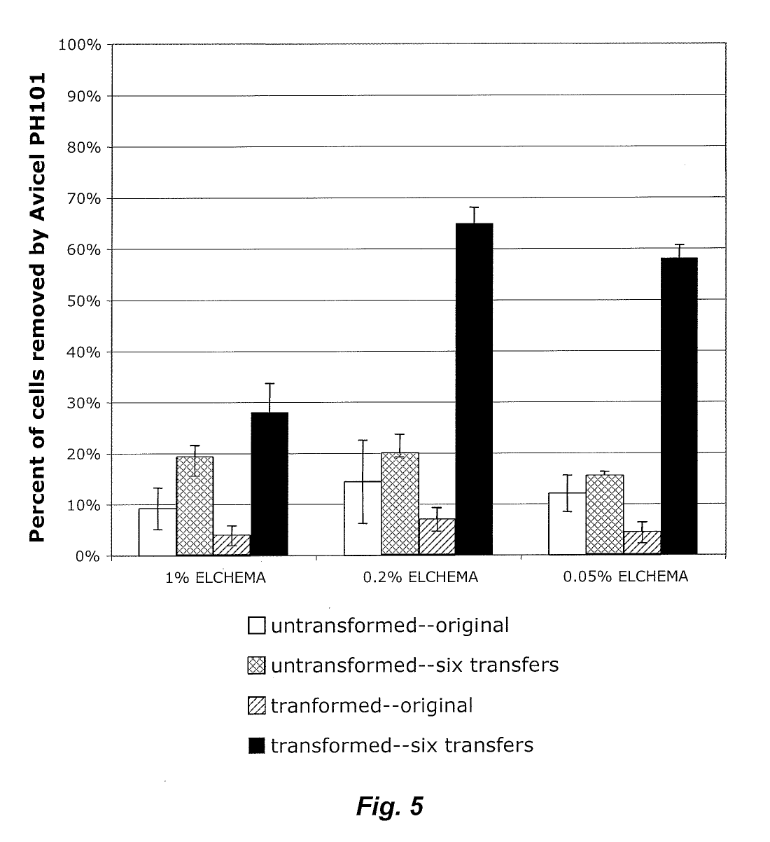

The cellulose binding results for two strains, which were subjected to the washing and re-growth procedure six times with a variety of starting ELCHEMA concentrations are summarized in FIG. 5. Of particular note is that strains with high OD(600) reductions by cellulose were obtained for strains with cellulases expressed when selected with 0.2 or 0.05% ELCHEMA, while untransformed strains increased their binding ability to a lesser degree. For the transformed strains expressing the cellulases, OD(600) reductions were increased by 5.5, 12.7, and 11.3 fold for the 1%, 0.2%, and 0.05% ELCHEMA concentrations used during selection, respectively. By comparison, the untransformed control increased its OD(600) reduction ability by only 1.6, 1.7, and 1.3 fold under the same conditions. These results demonstrate the increased cellulose binding ability of the transformed populations.

For comparison, the highest OD(600) reductions reported for Avicel are: 24.2% in Nam et al. and ˜23% in Ito et al. (24 hour, 4° C. incubation). Fukuda, T.; Ishikawa, T.; Ogawa, M.; Shiraga, S.; Kato, M.; Suye, S.; Ueda, M. “Enhancement of Cellulase Activity by Clones Selected from the Combinatorial Library of the Cellulose-Binding Domain by Cell Surface Engineering”, Biotechnology Progress 22: 933-938 (2006) do not report the percent OD(600) reduction for their strains, but indicate that their techniques have increased the strains binding capability by 1.5 fold, as compared to the 12.7 fold improvement observed with the present strains.

### Example 5

### Saccharomyces cerevisiae Strains with Tethered Cellulase Enzymes Capable of Growing in Semi-Continuous Culture with Avicel PH105

Endoglucanase I (EGI), cellobiohydrolase I (CBHI) and cellobiohydrolase II (CBHII) from Trichoderma reesei, along with β-glucosidase I (BGLI) from Saccharomycopsis fibuligera, were expressed as tethered proteins to the Saccharomyces cerevisiae cell surface by fusion with the C-terminal portion of cwp2 from S. cerevisiae, as described above.

Semi-continuous cultures of Saccharomyces cerevisiae strain CEN.PK 113-11C (both untransformed and transformed with BGLI, EGI, CBHI and CBHII) were carried out in 3 L (total volume) Applikon bioreactors. Avicel (˜20 g/L; PH105 from FMC Biopolymer, Philadelphia, Pa.) was added to synthetic complete medium for yeast (yeast nitrogen base without amino acids 1.7 g/L, ammonium sulfate 5 g/L, and supplemented with amino acids) lacking a carbon source. Avicel containing media was stirred in a 5 L carboy and intermittently pumped (every 80 minutes) into two side-by-side Applikon reactor systems, with working volumes of 1.8 L. The reactors were stirred at 400 rpm, and media was pumped out after a feeding following a 2 minute delay. Pump control, pH control and temperature control were all carried out using a DeltaV control system from Emerson Process Management, St. Louis, Mo. Conditions in the reactors were maintained at pH 5.0 using 1N HCl and 2N KOH, stirring at 400 rpm, an aeration rate of 1 VVM, and a temperature of 30° C. The dilution rate was maintained at ˜0.01 hr̂−1, which was verified by measuring the volume of the media accumulated in a waste carboy. The total dry weight of a system containing only water and avicel was monitored to verify that avicel was fed evenly over time. Inoculation cultures were pre-grown in YPD (yeast extract 10 g/L, peptone 20 g/L, glucose 20 g/L) and washed once with Tris-HCl buffer (pH 7.5) prior to inoculation. Cells were quantified by direct counts and dilution plating on YPD, as described above.

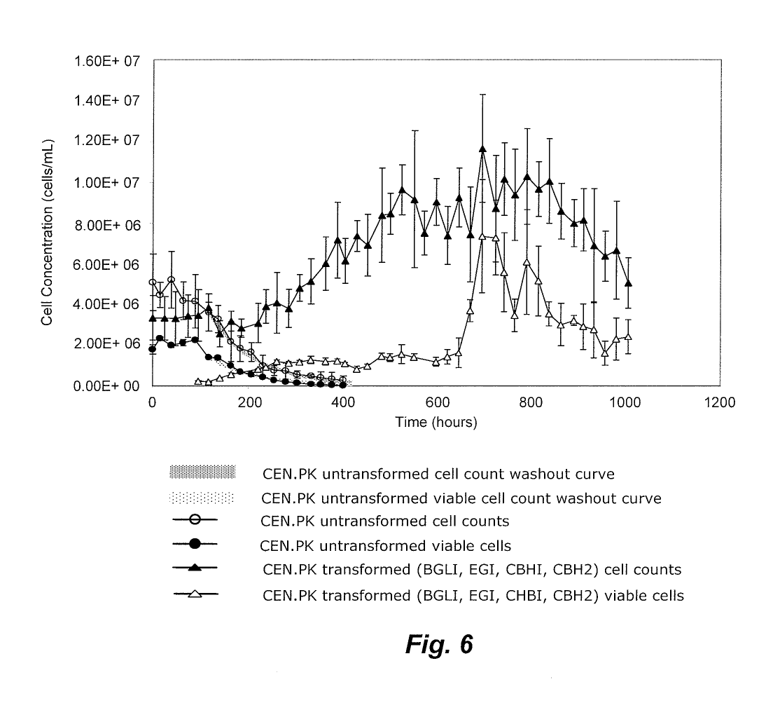

FIG. 6 shows the results from the two side-by-side reactors. The untransformed strain showed decreasing cell counts and viable cell counts over time, as expected in the absence of replication. Dotted lines show calculated wash-out (dilution) curves for non-replicating cells at the dilution rate measured. The observed correlation between the data and calculated wash-out curves confirms that the untransformed CEN.PK strain cannot replicate in the tested media.

On the other hand, the transformed strain of CEN.PK, expressing all four cellulase enzymes, grew and maintained its cell concentration for the duration of the continuous culture experiment (˜1000 hrs). In fact, the transformed strain showed a modest increase in cell concentration over the course of the experiment as measured both by cell counts and viable cell counts.

### Deposit of Recombinant Yeast Strains

Y294 and CEN.PK yeast strains containing the cellulase genes BGLI, EGI, CBHI and CBHII have been deposited with the American Type Culture Collection, Manassas, Va. 20110-2209. The deposits were made on Nov. 21, 2007 and received Patent Deposit Designation Numbers PTA-XXXX and PTA-XXXX, respectively. These deposits were made in compliance with the Budapest Treaty requirements that the duration of the deposits should be for thirty (30) years from the date of deposit or for five (5) years after the last request for the deposit at the depository or for the enforceable life of a U.S. patent that matures from this application, whichever is longer. The deposits will be replenished should one or more of them become non-viable at the depository.

The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or terminology employed herein is for the purpose of description and not limitation.

All references mentioned in this application are incorporated by reference to the same extent as though fully replicated herein.

What is claimed is:

1. A transformed yeast cell that expresses a plurality of genes, wherein the genes code for expression of tethered enzymes including endoglucanase, cellobiohydrolase and β-glucosidase.

2. The yeast according to claim 1, wherein the yeast is a member of the Saccharomyces genus.

3. The yeast according to claim 1, wherein the yeast is selected from the group consisting of Saccharomyces cerevisiae, Schizosaccharomyces pombe, Candida albicans, Kluyveromyces lactis, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Kluyveromyces marxianus, Issatchenkia orientalis and Schwanniomyces occidentalis.

4. The yeast according to claim 1, wherein the yeast is Saccharomyces cerevisiae.

5. The yeast according to claim 1, wherein the genes code for endoglucanse I (EGI), cellobiohydrolase I (CBHI), cellobiohydrolase II (CBHII) and β-glucosidase I (BGLI).

6. A method for selecting a transformed yeast cell with enhanced binding affinity for insoluble cellulose, comprising:
transforming a native organism to produce the yeast of claim 1, to produce a transformed yeast host;
culturing the transformed yeast host under suitable conditions for a period sufficient to allow growth and replication of the transformed yeast host;
exposing a sample of transformed yeast host from the culture to the insoluble cellulose; and
selecting the sample of transformed yeast host that provides at least a two fold reduction in supernatant optical density relative to a similarly cultured and exposed sample of the native organism.

7. A method for producing ethanol, said method comprising:
transforming a native organism to produce the yeast of claim 1, to produce a transformed yeast host; and
culturing the transformed yeast host in medium that contains cellulose under suitable conditions for a period sufficient to allow saccharification and fermentation of the cellulose to ethanol.

8. The method according to claim 7, wherein the yeast host is a member of the Saccharomyces genus.

9. The method according to claim 7, wherein the yeast host is selected from the group consisting of Saccharomyces cerevisiae, Schizosaccharomyces pombe, Candida albicans, Kluyveromyces lactis, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Kluyveromyces marxianus, Issatchenkia orientalis and Schwanniomyces occidentalis.

10. The method according to claim 7, wherein the yeast is Saccharomyces cerevisiae.

11. The method according to claim 7, wherein the genes code for endoglucanase I (EGI), cellobiohydrolase I (CBHI), cellobiohydrolase II (CBHII) and β-glucosidase I (BGLI).

12. A transformed organism, comprising,
a yeast that in a native state lacks the ability to saccharify cellulose, wherein the yeast is transformed with heterologous polynucleotides that express a plurality of enzymes that confer upon the yeast the ability to saccharify crystalline cellulose.

13. The yeast according to claim 12, wherein the yeast is a member of the Saccharomyces genus.

14. The yeast according to claim 12, wherein the yeast is selected from the group consisting of Saccharomyces cerevisiae, Schizosaccharomyces pombe, Candida albicans, Kluyveromyces lactis, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Kluyveromyces marxianus, Issatchenkia orientalis and Schwanniomyces occidentalis.

15. The yeast according to claim 12, wherein the yeast is Saccharomyces cerevisiae.

16. The yeast according to claim 12, wherein the polynucleotides code for the expression of at least one endoglucanase, at least one cellobiohydrolase and at least one β-glucosidase.

17. The yeast according to claim 16, wherein the endoglucanase, cellobiohydrolase and β-glucosidase are tethered to the yeast cell surface.

18. The yeast according to claim 12, wherein the polynucleotides code for endoglucase I (EGI), cellobiohydrolase I (CBHI), cellobiohydrolase II (CBHII) and β-glucosidase I (BGLI).

19. A method for selecting a transformed yeast cell with enhanced binding affinity for insoluble cellulose, comprising:
transforming a native organism to produce the yeast of claim 12, to produce a transformed yeast host;
culturing the transformed yeast host under suitable conditions for a period sufficient to allow growth and replication of the transformed yeast host;
exposing a sample of transformed yeast host from the culture to the insoluble cellulose; and
selecting the sample of transformed yeast host that provides at least a two fold reduction in supernatant optical density relative to a similarly cultured and exposed sample of the native organism.

20. A method for producing ethanol, said method comprising:
transforming a native organism to produce the yeast of claim 12, to produce a transformed yeast host; and
culturing the transformed yeast host in medium that contains cellulose under suitable conditions for a period sufficient to allow saccharification and fermentation of the cellulose to ethanol.

21. The method according to claim 20, wherein the yeast host is a member of the Saccharomyces genus.

22. The method according to claim 20, wherein the yeast host is selected from the group consisting of Saccharomyces cerevisiae, Schizosaccharomyces pombe, Candida albicans, Kluyveromyces lactis, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Kluyveromyces marxianus, Issatchenkia orientalis and Schwanniomyces occidentalis.

23. The method according to claim 20, wherein the yeast is Saccharomyces cerevisiae.

24. The method according to claim 20, wherein the polynucleotides code for the expression of at least one endoglucanase, at least one cellobiohydrolase and at least one β-glucosidase.

25. The yeast according to claim 24, wherein the endoglucanase, cellobiohydrolase and β-glucosidase are tethered to the yeast cell surface.

26. An isolated polynucleotide comprising:
(a) a polynucleotide sequence of SEQ ID NO: 11;
(b) a polynucleotide sequence of SEQ ID NO: 12;
(c) a polynucleotide sequence of SEQ ID NO: 28;
(d) a polynucleotide sequence of SEQ ID NO: 29; and
(e) a polynucleotide sequence of SEQ ID NO: 30; or
(f) a polynucleotide sequence having at least about 90% sequence identity with the polynucleotide sequences of (a)-(e).

27. The polynucleotide of claim 26, having about 95% sequence identity with the polynucleotide sequences of (a)-(e).

28. A vector comprising the isolated polynucleotide of claim 27.

29. A host cell genetically engineered to express a compliment of the polynucleotide of claim 27.

30. The host cell of claim 29, wherein the host cell is a yeast cell.

31. A method of producing ethanol, comprising:
culturing a yeast host cell according to claim 29 in medium containing cellulose under suitable conditions for a period of time sufficient to allow saccharification and fermentation of the cellulose to ethanol.

32. The method according to claim 31, wherein the yeast host cell is a member of the Saccharomyces genus.

33. The method according to claim 31, wherein the yeast host cell is selected from the group consisting of Saccharomyces cerevisiae, Schizosaccharomyces pombe, Candida albicans, Kluyveromyces lactis, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Kluyveromyces marxianus, Issatchenkia orientalis and Schwanniomyces occidentalis.

34. The method according to claim 31, wherein the yeast host cell is Saccharomyces cerevisiae.

35. A genetic construct comprising SEQ ID NO: 11, SEQ ID NO: 12, SEQ ID NO: 28, SEQ ID NO: 29 and SEQ ID NO: 30 operably connected to promoters expressible in yeast.

36. A recombinant yeast comprising the genetic construct of claim 35.

37. The recombinant yeast of claim 36 comprising Saccharomyces cerevisiae.
